SEQUENCE LISTING (56 sequences)

SEQ ID NO: 1 - protein, 243 aa, Sus scrofa
  misc_feature (69)..(69): Xaa can be any naturally occurring amino acid
MLLLGAVLLLLALPSLGQETTEKPGALLPMPKGACAGWMAGIPGHPGHNGTPGRDGRDGVPGEKGEKGXTGLTXPKGDTGESGVTGVEGPRGFPGIPGRKGEPGESAYVYRSAFSVGLETRVTVPNMPIRXTKIFYNQQNHYDVTTGKFHCNIPGLYYFSFHVTVYLKDVKVSLYKKDKAVLFTYDQYQDKNVDQASGSVLLYLEKGDQVWLQAYGDEENNGVYADNVNDSIFTGFLLYHNIE

SEQ ID NO: 2 - protein, 225 aa, Sus scrofa
MLLLGAVLLLLALPSLGQETTEKPGALLPMPKGACAGWMAGIPGHPGHNGTPGRDDRDGVPGEKGEKGDTGLTGPKGDTGESGVTGVEGPRGFPGIPGRKGEPGESAYVYRSAFSVGLETRVTVPNMPIRFTKIFYNQQNHYDVTTGKFHCNIPGLYYFSFHITVYLKDARVSLYKKDKAVLFTYDQYQDKNVDQASGSVLLYLEKGDQVWLQAYGDEENNGVYA

SEQ ID NO: 3 - protein, 145 aa, Sus scrofa
PKGACAGWMAGIPGHPGHNGTPGRDGRDGVPGEKGEKGDTGLTGPKGDTGESGVTGVEGPRGFPGIPGRKGEPGESAYVYRSAFSVGLETRVTVPNMPIRFTKIFYNQQNHYDVTTGKFHCNIPGLYYFSFHITVYLKDVKVSLF

SEQ ID NO: 4 - protein, 144 aa, Sus scrofa
DGRDGVPGEKGEKGDTGLTGPKGDTGESGVTGVEGPRGFPGIPGRKGEPGGSAYVYRSAFSVGLETRVTVPNMPIRFTKIFYNQQNHYDVTTGKFHCNIPGLYYFSFHITVYLKDVKVSLYKKDKAVLFTYDQYQDKNVDQASG

SEQ ID NO: 5 - protein, 244 aa, Oryctolagus cuniculus
MLLLQAVLLLLALPSHGQDSTTESPGVLIPAPKGACAGWIAGIPGHPGHNGTPGRDGRDGTPGEKGEKGDAGLVGPKGDTGETGVTGAEGPRGFPGSPGRKGEPGEGAYVYRSAFSVGLEGRVTIPNVPIRFTKIFYNQQNHYDSTTGKFRCNIPGLYYFSYHITVYMKDVKVSLFKKDKAMLFTYDQYQDKNVDQASGSVLLHLQVDDQVWLQVYGDGDHNGLYADNVNDSISTGFLLYHDTN

SEQ ID NO: 6 - protein, 244 aa, Homo sapiens
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 7 - protein, 245 aa, Anser anser
MRDSVGFLLCSLLLVAPCCTEVAAPDPQPDPKTPCANWMGGAPGYPGHNGLPGRDGKDGKDGLKGEKGEQGLQGSKGDQGEKGTPGPEGPRGFPGYPGLKGDKGEGAYVYRSAFSVGLTERAPHPNVPIRFSKIFYNEQNHYDASTGKFLCSIPGMYYFAYHLTVYMSDVKVSLYKKDKAVIFTYDQFQTNNVDQASGSVLLHLSSGDEVWLQVYGEGDNNGVYADNINDSTFMGFLLYPDMDFH

SEQ ID NO: 8 - protein, 244 aa, Canis lupus
MLLLRAVLLLLVLPAHGQDSVAEGPGVLLPLPKGACPGWMAGIPGHPGHNGTPGRDGRDGTPGEKGEKGDAGLVGPKGDTGETGVTGVEGPRGFPGTPCRKGEPGESAYVHRSAFSVGLESRITVPNVPIRFTKIFYNLQNHYDGTTGKFHCNIPGLYYFSYHITVYLKDVKVSLYKKDKAMLFTYDQYQEKNVDQASGSVLLHLEVGDQVWLQVYGDGDSYGIYADNVNDSTFTGFLLYHDTN

SEQ ID NO: 9 - protein, 243 aa, Macaca fuscata
MLLGAVLLLLALPSHGQDTTTQGPGVLLPLPKGPCTGWMAGIPGHPGHNGVPGRDGRDGTAGEKGEKGDPGLIGPKGDTGETGVTGAEGPRGFPGIQGRKGEPGEGAYVYRSAFSVGLETYVTVPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 10 - protein, 243 aa, Macaca mulatta
MLLGAVLLLLALPSHGQDTTTQGPGVLLPLPKGACTGWMAGIPGHPGHNGVPGRDGRDGTPGEKGEKGDPGLIGPKGDTGETGVTGAEGPRGFPGIQGRKGEPGEGAYVYRSAFSVGLETYVTVPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 11 - protein, 244 aa, Gallus gallus
MRGSVGFLLCSLLLALSGTEMADQADQSDPKMSCANWMGGAPGHPGHNGLPGRDGKDGKDGQKGDKGEPGLQGVKGGTGEKGATGAEGPRGFPGHMGMKGQKGESSYVYRSAFSVGLTERAPHPNVPIRFTKIFYNEQNHYDSSTGKFLCSIPGTYFFAYHLTVYMTDVKVSLYKKDKAVIFTYDQFQENNVDQASGSVLLHLSLGDEVWLQVYGEGNNNGVYADNINDSTFMGFLLYPDTDDR

SEQ ID NO: 12 - protein, 244 aa, Felis catus
MLLLRAVLLLLVLPIRGQDSETEGPGVVVPLPKGACTGWMAGIPGHPGHNGTPGRDGRDGTPGEKGEKGDPGLVGPKGDTGETGVTGIEGPRGFPGIPGRKGEPGESAYVYRSAFSVGLESRVTVPNVPIRFTKIFYNQQNHYDVTTRKFHCNIPGLYYFSYHITVYLKDVKVSLYKRDKAMLFTYDQYQEKNVDQASGSVLLHLETGDEVWLQVYGDGDYNGLYADNVNDSTFTGFLLYYDTV

SEQ ID NO: 13 - protein, 165 aa, Felis catus
PGVLLPLPKGACTGWMAGIPGHPGHNGTPGRDGRDGTPGEKGEKGDPGLVGPKGDTGETGVTGIEGPRGFPGIPGRKGEPGESAYVYRSAFSVGLESRVTVPNVPIRFTKIFYNQQNHYDVTTGKFHCNIPGLYYFSYHITVYLKDVKVSLYKRDKAMLFTYDQY

SEQ ID NO: 14 - protein, 106 aa, Felis catus
TGVTGIEGPRGFPGIPGRKGEPGESAYVYRSAFSVGLESRVTVPNVPIRFTKIFYNQQNHYDVTTGKFHCNIPGLYYFSYHITVYLKDVKVSLYKRDKAMLFTYDQ

SEQ ID NO: 15 - protein, 245 aa, Anas platyrhynchos
MRDSAGFLLCSLLLVAPHCTEVAAQDPQPDPKTPCANWMGGAPGYPGHNGLPGRDGKDGKDGLKGEKGEQGLQGSKGDQGAMGSAGPEGPRGFPGQPGLKGDKGEGAYVYRSAFSVGLTERAPHPNVPIRFSKIFYNEQNHYDASTGKFLCSIPGTYYFAYHLTVYMSDVKVSLYKKDKAVIFTYDQFQTNNIDQASGSVLLHLSSGDEVWLQVYGEGENNGVYADNINDSTFMGFLLYPDMDFH

SEQ ID NO: 16 - protein, 229 aa, Vulpes vulpes
LLLVLPAHGQDSVAEGPGVLLPLPKGACPGWMAGIPGHPGHNGTPGRDGRDGTPGEKGEKGDPGLVGPKGDTGETGITGVEGPRGFPGTPGRKGEPGESAYVHRSAFSVGLESRITVPNVPIRFTKIFYNLQNHYDGTTGKFHCNIPGLYYFSYHITVYLKDVKVSLYKKDKAMLFTYDQYQEKNVDQASGSVLLHLEVGDQVWLQVYGDGDSYGIYADNVNDSTFTGF

SEQ ID NO: 17 - protein, 229 aa, Alopex lagopus; identical to SEQ ID NO: 16

SEQ ID NO: 18 - protein, 229 aa, Nyctereutes procyonoides; identical to SEQ ID NO: 16

SEQ ID NO: 19 - protein, 244 aa, Rattus norvegicus
MLLLQALLFLLILPSHEGITATEGPGALVPPPKETCAGWMAGIPGYPGHNGIPGRDGRDGTPGEKGEKGDAGVLGPKGDPGDAGMTGAEGPRGFPGTPGRKGEPGEAAYMYHSAFSVGLETRVTVPNVPIRFTKIFYNQQNHYDGSTGKFHCNIPGLYYFSYHITVYMKDVKVSLFKKDKAVLFTYDQYQEKNVDQASGSMLLHLEVGDQVWLQVYGEGDNNGLYADNVNDSTFTGFLLYHDTN

SEQ ID NO: 20 - protein, 247 aa, Mus musculus
MLLLQALLFLLILPSHAEDDVTTTEELAPALVPPPKGTCAGWMAGIPGHPGHNGTPGRDGRDGTPGEKGEKGDAGLLGPKGETGDVGMTGAEGPRGFPGTPGRKGEPGEAAYVYRSAFSVGLETRVTVPNVPIRFTKIFYNQQNHYDGSTGKFYCNIPGLYYFSYHITVYMKDVKVSLFKKDKAVLFTYDQYQEKNVDQASGSVLLHLEVGDQVWLQVYGDGDHNGLYADNVNDSTFTGFLLYHDTN

SEQ ID NO: 21 - protein, 240 aa, Bos taurus
MLLQGALLLLLALPSHGEDNMEDPPLPKGACAGWMAGIPGHPGHNGTPGRDGRDGTPGEKGEKGDPGLVGPKGDTGETGITGIEGPRGFPGTPGRKGEPGESAYVYRSAFSVGLERQVTVPNVPIRFTKIFYNQQNHYDGTTGKFLCNIPGLYYFSYHITVYLKDVKVSLYKNDKALLFTHDQFQDKNVDQASGSVLLYLEKGDQVWLQVYEGENHNGVYADNVNDSTFTGFLLYHNIVE

SEQ ID NO: 22 - protein, 275 aa, Danio rerio
MSLLRTIILGIILTGHLCGGQDELLEDSLETAVDEVATEGIEELEEPEEEGLDLSPPDDRQPCAMWMGGVPGTPGHSGKPGRDGRDGRDGPRGEKGDQGEAGEKGDPGEKGDIGNAGPRGFPGNPGLKGARGESASSYHSAFSVGLSEIVSATNVPIRFNKFFYNDQHHYDDVSGKFRCVLPGVYFFTYHLTVYTKDAKVSLYKNDKAIMFTYDQYQETNVDQASGSVILRLEAGDEVWLQVYGDETVGGVYADNTNDSTFSGFLLYPVNPAERR

SEQ ID NO: 23 - protein, 220 aa, Danio rerio
VARLGFSQIDLAEQNSREPCARWMRGVSGTPGFGGIPGRDGRDGREGEKGDNGEPGPKGPTGEPGKPGDEGFPGKRGFPGNPGLKGESGEASFPYHSAFSMGLTDKVSPASGSPIRFTKTFYNEQHHYDDISGKFRCAIPGIYYFTYHLTINGKETKVAMFRNGRTVAFTLDQFHSGNLDQASGGVILNLSSGDEVWLQLYDDIFDEGIYVDYNNDSTFS

SEQ ID NO: 24 - protein, 200 aa, Danio rerio
PCAMWMGGVPGTPGHSGKPGRDGRDGRDGQRGEKGDQGEAGEKGDPGEKGDIGNAGPRGFPGNPGLKGARGESASSYHSAFSVGLSEIVSATNVPIRFNKFFYNDQHHYDDVSGKFRCVLPGVYFFTYHLTVYTKDAKVSLYKNDKAIMFTYDQYQETNVDQASGSVILRLEAGDEVWLQVYGDETVGGVYADNTNDSTF

SEQ ID NO: 25 - protein, 484 aa, Danio rerio
MTLRFRFVAFECIFEWESMGGKAQCDRCLKLIPLVREHIQERSKIGEGSGCRLGAVESVLHPMLFNVLTNPTRHSVVSLISLAVLSYERYCTMMGSTQADSTNYRKVVIGIAFSWIYSMVWTLPPLFGWSCYGPEGPGTTCSVNWAARTPNNVSYIVCLFVFCLILPFIVIVYSYGRLLQAITQLLRLSGQIFLGMTSREETRTQGQRSTSYQYTQKDPLLMNSLTYRLLLPLMSSAWIVVVCVAMVARLGFSQIDLAEQNSREPCARWMRGVSGTPGFGGIPGRDGRDGREGEKGDNGEPGPKGPTGEPGKPGDEGFPGKRGFPGNPGLKGESGEASFPYHSAFSMGLTDKVSPASGSPIRFTKTFYNEQHHYDDISGKFRCAIPGIYYFTYHLTINGKETKVAIFRNGRTVAFTLDQFHSGNLDQASGGVILNLSSGDEVWLQLYDDIFDEGIYVDYNNDSTFSGFLLTPKVLSNSFDNRKR

SEQ ID NO: 26 - protein, 249 aa, Ornithorhynchus anatinus
MKQGPYQLLGFLLLANLCYSQVGPTEESADDPRFPKGHCAGWMGGAPGHPGHNGAPGRDGRDGTNGEKGEKGDPGLEGSKGDPGEIGVKGIEGPRGFPGNPGKKGDRGEGAYVYRSAFSVGLVSGVPVPNIPIKFTKIFYNNQNHYDPTTGKFHCNLPGLYYFSYHITVYTKDVKVSLYKKDRAVMFTFDQFQQNNVDQASGSVLLHLDAGEEVWLQVYGEGEHNGIYADNVNDSTFTGFLLYPDMDHE

SEQ ID NO: 27 - protein, 738 aa, Homo sapiens
  SIGNAL (1)..(24): signal sequence
MTSSGPGPRFLLLLPLLLPPAASASDRPRGRDPVNPEKLLVITVATAETEGYLRFLRSAEFFNYTVRTLGLGEEWRGGDVARTVGGGQKVRWLKKEMEKYADREDMIIMFVDSYDVILAGSPTELLKKFVQSGSRLLFSAESFCWPEWGLAEQYPEVGTGKRFLNSGGFIGFATTIHQIVRQWKYKDDDDDQLFYTRLYLDPGLREKLSLNLDHKSRIFQNLNGALDEVVLKFDRNRVRIRNVAYDTLPIVVHGNGPTKLQLNYLGNYVPNGWTPEGGCGFCNQDRRTLPGGQPPPRVFLAVFVEQPTPFLPRFLQRLLLLDYPPDRVTLFLHNNEVFHEPHIADSWPQLQDHFSAVKLVGPEEALSPGEARDMAMDLCRQDPECEFYFSLDADAVLTNLQTLRILIEENRKVIAPMLSRHGKLWSNFWGALSPDEYYARSEDYVELVQRKRVGVWNVPYISQAYVIRGDTLRMELPQRDVFSGSDTDPDMAFCKSFRDKGIFLHLSNQHEFGRLLATSRYDTEHLHPDLWQIFDNPVDWKEQYIHENYSRALEGEGIVEQPCPDVYWFPLLSEQMCDELVAEMEHYGQWSGGRHEDSRLAGGYENVPTVDIHMKQVGYEDQWLQLLRTYVGPMTESLFPGYHTKARAVMNFVVRYRPDEQPSLRPHHDSSTFTLNVALNHKGLDYEGGGCRFLRYDCVISSPRKGWALLHPGRLTHYHEGLPTTWGTRYIMVSFVDP

SEQ ID NO: 28 - protein, 738 aa, Homo sapiens; identical to SEQ ID NO: 27

SEQ ID NO: 29 - protein, 739 aa, Bos taurus
MASGPELRPLLLLLLLLSPSPAASASDRPRGSDPVNPEKMLVITVATAETEGYRRFLQSAEFFNYTVRTLGLGEEWRGGDVARTVGGGQKVRWLKKEMEKYAEREDMVIMFVDSYDVVLAGSPSELLKKFVQSGSRLLFSAESFCWPEWGLAEQYPEVGTGKRFLNSGGFIGFAPTIHQIVRQWKYKDDDDDQLFYTRLYLDPGLREKLGLSLDHKSRIFQNLNGALDEVVLKFGRNRVRIRNVAYDTLPVVVHGNGPTKLQLNYLGNYVPNGWTPEGGCGFCNQGRRPLPGGQPPPRVLLAVFVEQPTPFLPRFLQRLLLLDYPPDRVTLFLHNNEVYHEPHIDESWPQLQDHFSAVKLVGPEEALTPGEARDMAMDICRQDPKCEFYFSLDADTVITNPQTLRILIEANRKVIAPMLSRHGKLWSNFWGALSPDEYYARSEDYVELVQRKRVGVWNVPYISQAYVIRGETLRTELPQREVFSGSDTDPDMAFCKSLRDKGIFLHLSNQHEFGRLLATSRYDTDHLHPDLWQIFDNPLDWKEQYIHENYTRALEGEGLVEQPCPDVYWFPLLSEQMCDELVEEMEHYGQWSGGRHEDSRLAGGYENVPTVDIHMKQVGYEDQWLQLLRTYVGPMTESLFPGYHTKTRAVMNFVVRYRPDEQPSLRPHHDSSTFTLNVALNHKGLDYEGGGCRFLRYDCVISSPRKGWGLLHPGRLTHYHEGLPTTRGTRYIMVSFVDP

SEQ ID NO: 30 - protein, 740 aa, Canis lupus
MASSGPGLRLLLGLLLLLPPPPATSASDRPRGGDPVNPEKLLVITVATAETEGYRRFLWSAEFFNYTVRTLGLGEEWRGGDVARTVGGGQKVRWLKKEMEKYADREDMVIMFVDSYDVILAGSPAELLKKFVQSGSRLLFSAEGFCWPEWGLAEQYPEVGTGKRFLNSGGFIGFAPTIHKVVRQWKYKDDDDDQLFYTRLYLDPGLREKLSLNLDHKSRIFQNLNGALDEVVLKFDRNRVRIRNVAYDTLPVVVHGNGPTKLHLNYLGNYVPNGWTPQGGCGFCGRDRRTLPGGQPPPRVLLAVFVEQPTPFLPRFLQRLLLLDYPPDRVTLFLHNNEVYHEPHIADSWPQLQDHFSAVKLVGPEEALTPGEARDMAMDSCRQDPECEFYFSLDADAVITNPQTLRILIEENRKVIAPMLSRHGKLWSNFWGALSPDEYYARSEDYVELVQRKRVGVWNVPYISQAYVIRGETLRTELPQREVFSGSDTDPDMAFCKSLRDKGIFLHLSNQHEFGRLLATSRYDTDHLHPDLWQIFDNPLDWKEQYIHENYSRALEGEGLVEQPCPDVYWFPLLSDQMCDELVEEMEHYGQWSGGRHEDSRLAGGYENVPTVDIHMKQVGYEDQWLQLLRTYVGPMTESLFPGYHTKTRAVMNFVVRYRPDEQPSLRPHHDSSTFTLNVALNHKGLDYEGGGCRFLRYDCIVSSPRKGWGLLHPGRLTHYHEGLPTTRGTRYIMVSFVDP

SEQ ID NO: 31 - protein, 754 aa, Canis lupus
MASSGPGLRLLLGLLLLLPPPPATSASDRPRGGDPVNPEKLLVITVATAETEGYRRFLWSAEFFNYTVRTLGLGEEWRGGDVARTVGGGQKVRWLKKEMEKYADREDMVIMFVDSYDVILAGSPAELLKKFVQSGSRLLFSAEGFCWPEWGLAEQYPEVGTGKRFLNSGGFIGFAPTIHKVVRQWKYKDDDDDQLFYTRLYLDPGLREKLSLNLDHKSRIFQNLNGALDEVVLKFDRNRVRIRNVAYDTLPVVVHGNGPTKLHLNYLGNYVPNGWTPQGGCGFCGRDRRTLPGGQPPPRVLLAVFVEQPTPFLPRFLQRLLLLDYPPDRVTLFLHNNEVYHEPHIADSWPQLQDHFSAVKLVGPEEALTPGEARDMAMDSCRQDPECEFYFSLDADAVITNPQTLRILIEENRKVIAPMLSRHGKLWSNFWGALSPDEYYARSEDYVELVQRKRVGVWNVPYISQAYVIRGETLRTELPQREVFSGSDTDPDMAFCKSLRDKVTTHVSSHPPPPLQGIFLHLSNQHEFGRLLATSRYDTDHLHPDLWQIFDNPLDWKEQYIHENYSRALEGEGLVEQPCPDVYWFPLLSDQMCDELVEEMEHYGQWSGGRHEDSRLAGGYENVPTVDIHMKQVGYEDQWLQLLRTYVGPMTESLFPGYHTKTRAVMNFVVRYRPDEQPSLRPHHDSSTFTLNVALNHKGLDYEGGGCRFLRYDCIVSSPRKGWGLLHPGRLTHYHEGLPTTRGTRYIMVSFVDP

SEQ ID NO: 32 - protein, 741 aa, Mus musculus
MAAAGPEPRLLLLLLLLLPPLPPVTSASDRPRGANAVNPDKLLVITVATAETEGYRRFLQSAEFFNYTVRTLGLGQEWRGGDVARTVGGGQKVRWLKKEMEKYADQKDMIIMFVDSYDVILASSPTELLKKFVQSGSHLLFSAESFCWPEWGLAEQYPEVGMGKRFLNSGGFIGFAPTIHQIVRQWNYKDDDDDQLFYTQLYLDPGLREKLKLSLDHKSRIFQNLNGALDEVILKFDQNRVRIRNVAYDTLPVVVHGNGPTKLQLNYLGNYVPNGWTPQGGCGFCNQTLRTLPGGQPPPRVLLAVFVEQPTPFLPRFLQRLLLLDYPPDRISLFLHNSEVYHEPHIADAWPQLQDHFSAVKLVGPEEALSAGEARDMAMDSCRQNPECEFYFSLDADAVLTNPETLRVLIEQNRKVIAPMLSRHGKLWSNFWGALSPNEYYARSEDYVELVQRKRVGVWNVPYISQAYVIRGETLRTELPQKEVFSSSDTDPDMAFCKSVRDKGIFLHLSNQHEFGRLLATSRYDTDHLHPDLWQIFDNPVDWREQYIHENYSRALDGEGLVEQPCPDVYWFPLLTEQMCDELVEEMEHYGQWSGGRHEDSRLAGGYENVPTVDIHMKQVGYEDQWLQLLRTYVGPMTEYLFPGYHTKTRAVMNFVVRYRPDEQPSLRPHHDSSTFTLNVALNHKGVDYEGGGCRFLRYDCRISSPRKGWALLHPGRLTHYHEGLPTTRGTRYIMVSFVDP

SEQ ID NO: 33 - protein, 741 aa, Rattus norvegicus
  SIGNAL (1)..(27): signal sequence
MAASVPEPRLLLLLLLLLPPLPPVTSASDRPRGANPVNPDKLLVITVATAETEGYRRFLQSAEFFNYTVRTLGLGQEWRGGDVARTVGGGQKVRWLKKEMEKYASQEDMIIMFVDSYDVILASSPTELLKKFVQSGSHLLFSAESFCWPDWGLAEQYPEVGVGKRFLNSGGFIGFAPTIHRIVRQWKYKDDDDDQLFYTQLYLDPGLREKLKLSLDHKSRIFQNLNGALDEVVLKFDQNRVRIRNVAYDTLPVVVHGNGPTKLQLNYLGNYVPNGWTPQGGCGFCNLNRRTLPGGQPPPRVLLAVFVEQPTPFLPRFLQRLLLLDYPPDRISLFLHNNEVYHEPHIADAWPQLQDHFSAVKLVGPEEALSSGEARDMAMDSCRQNPECEFYFSLDADAVLTNPETLRILIEQNRKVIAPMLSRHGKLWSNFWGALSPDEYYARSEDYVELVQRKRVGLWNVPYISQAYVIRGETLRTELPEKEVFSSSDTDPDMAFCRSVRDKGIFLHLSNQHEFGRLLSTSHYDTDHLHPDLWQIFDNPVDWREQYIHENYSRALDGEGLVEQPCPDVYWFPLLTEQMCDELVEEMEHYGQWSGGRHEDSRLAGGYENVPTVDIHMKQVGYEDQWLQLLRTYVGPMTEHLFPGYHTKTRAVMNFVVRYRPDEQPSLRPHHDSSTFTLNVALNHKGVDYEGGGCRFLRYDCRVSSPRKGWALLHPGRLTHYHEGLPTTRGTRYIMVSFVDP

SEQ ID NO: 34 - protein, 741 aa, Rattus norvegicus; identical to SEQ ID NO: 33

SEQ ID NO: 35 - protein, 738 aa, Pongo abelii
MTSSGPGPRFLLLLPLLLPPAASASDRPRGRDPVNPEKLLVITVATAETEGYLRFLRSAEFFNYTVRTLGLGEEWRGGDVARTVGGGQKVRWLKKEMEKYADREDMIIMFVDSYDVILAGSPTELLKKFVQSGSRLLFSAESFCWPEWGLAEQYPEVGTGKRFLNSGGFIGFATTIHQIVRQWKYKDDDDDQLFYTRLYLDPGLREKLSLNLDHKSRIFQNLNGALDEVVLKFDRNRVRIRNVAYDTLPVVVHGNGPTKLQLNYLGNYVPKGWTPEGGCGFCNQDRRTLPGGQPPPRVFLAVFVEQPTPFLPRFLQRLLLLDYPPDRVTLFLHNNEVFHEPHIADSWPQLQDHFSAVKLVGPEEALSPGEARDMAMDLCRQDPECEFYFSLDADTVLTNLQTLRILIEENRKVIAPMLSRHGKLWSNFWGALSPDEYYARSEDYVELVQRKRVGVWNVPYISQAYVIRGDTLRTELPQRDVFSGSDTDPDMAFCKSFRDKGIFLHLSNQHEFGRLLATSRYDTEHLHPDLWQIFDNPVDWKEQYIHENYSRALEGEGIVEQPCPDVYWFPLLSEQMCDELVAEMEHYGQWSGGRHEDSRLAGGYENVPTVDIHMKQVGYEDQWLQLLRTYVGPMTESLFPGYHTKARAVMNFVVRYRPDEQPSLRPHHDSSTFTLNVALNHKGLDYEGGGCRFLRYDCVISSPRKGWALLHPGRLTHYHEGLPTTWGTRYIMVSFVDP

SEQ ID NO: 36 - protein, 730 aa, Danio rerio
MTPVPVILTVILAVIQPCRTEPRKPNELLVITAATEVTDGYLRFMRTIRQFNYTIQVLGLGEQWRGGDVARTVGGGQKVRWLKTELEKHKDKQNTVIMFVDSYDVILASGPVELLRKFSRFSHRVVFSAEGFCWPDQRLASKYPAVHHGKRYLNSGGFIGFAPEIHAIVQQWKYKDDDDDQLFYTRIYLDKEKRRKFNMTLDHRSQIFQNLNGAIEEVVLKFEKSRVRVRNVAYDTLPVVIHGNGPTKLQLNYLGNYVPTAWTYENGCGICEEDLLDLSHLSDEEMPLVHVAVFIEQPMPFLEEFLERLATLNYPHTRIRLFLHNNVVYHEQHVERFWTRHRSLFTGARIVGPEENLKHDQARTMAVEACKKDVSCDYFFSLDADVALTNPDVLRILIEENKSVIAPMLSRHGKLWSNFWGALSPEGFYSRAEDYIDIVQSKRVGLWNVPYITQVYLIRGETLRSRLAAVSLYQQEGMDPDMSFCKSVREQGIFMFVSNRDEFGRLVSSANYNISRLHPDMWQIFDNPVDWREKYIHENYSRIFEDDESVVEQPCPDVYWFPAFSERMCDDLVETMEEFGQWSGGGHKDERLSGGYENVPTVDIHMNQIQFEKEWLKFLKEYIVPVTEKLYPGYYPKAQAVMNFVVRYRPDEQPSLRPHHDSSTFTINIALNSKGVDYEGGGCRFLRYNCKVESPRKGWSFMHPGRLTHYHEGLPTTQGTRYIMVSFVDP

SEQ ID NO: 37 - protein, 730 aa, Danio rerio; identical to SEQ ID NO: 36
  SIGNAL (1)..(20): signal sequence

SEQ ID NO: 38 - protein, 733 aa, Xenopus laevis
  SIGNAL (1)..(20): signal sequence
METRAALGLVLLVLCGLSLGESGRKEALRPDKLLVVTVATEATEGYLRFLRTARHFNYTVRTLGLGHEWKGGDVARTVGGGQKVRWLKHELEQHKDQDDLIIMFVDSYDVVISGSPTELLWKFQRFEHKVVFSAEGFCWPEWSLAESYPPITNGKRFLNSGGFIGFAPQLYQMVQLWKYKDNDDDQLFYTKIYLDESMREKFDITLDHKSNIFQNLNGAIDEVVLKFESNKVRARNVAYDTIPVVIHGNGPTKLQLNYLGNYVPNSWTHEGGCEVCDDDLLDLSMLEDDALPHVLLGVFIEQPTPFIPQFLQRLVQLDYPRNRLSLYIHNSEVYHERHIEVFYKKYKDSFTSIKIVGPEEAMSQGEARDMGMDLCRQDQTCDYYFSVDADVALTNPDTLYILIQENKKVIAPMVSRSGKLWSNFWGALSPEGYYARSEDYVDIVQAKRAGVWNVPYIAHVYLIKGETLRAELSNKNIFTLPQMDPDMSVCKSIRDKNVFLHISNRDEFGRLLSTSKYNTSRLHNDLWQIFENPVDWKEKYIHENYSKIFEEDYYQQPCPDVYWFPVFSEVMCDEFVEEMENFGQWSGGKNQDQRLAGGYENVPTVDIHMTQIGYQEEWLKFLQEYIAPVTEKLFPGYYTKAKALLNFIVRYRPDEQPSLRPHHDSSTFTVNIALNNKGIDYEGGGCRFLRYNCRVESPRKGWSFMHPGRLTHYHEGLPTTRGTRYIMVSFVDP

SEQ ID NO: 39 - protein, 731 aa, Takifugu rubripes
  SIGNAL (1)..(21): signal sequence
MFAGRLLSLLALTALHSAASAGRQSLSPENLLVITAATEETDGFNRFMRTAREFNYTVKVLGLGEEWRGGDVARTVGGGQKVRWLKKELSKHSDKENMVIMFVDSYDVILAAGPEEPLYKFSRLGHKVVFSAEGFCWPDQRLASKYPEVHSGKRYLNSGGFIGLASELSAIVQQWKYKDNDDDQLFYTRIYLDKVQRTKFNMTLDHRSRIFQNLNGAVDEVVLKFERSKVRARNVAYDTLPVVIHGNGPTKLQLNYLGNYVPTAWTFAGGCGICDDELRLLNEDEEMPLVHVGVFIEKATPFLEEFLERLTAMSYPTARLRLFIHNNVFYHERHIHRFWERHRALFLDAQLVGPEENLPESKARNMAAEACKKDPRCEFYFSIDSDVALTNPDTLRILIEENKSVIAPMLSQHGKLWSNFWGALSPEGYYSRSEDYIEIVQGKRIGLWNVPYITQVYLIKGSVLRSKLSQLSLFVDEEMDSDMVFCRNIRDQGIFLFVSNRDEFGRLVTSTNFNTSRLHPDMWQIFDNPLDWKEKYIHENYSKVFEEQESFVEQPCPDVYWFPAFSEKMCDHLVETMEDNGQWSSGGHRDERLSGGYENVPTVDIHMNQIGFEKEWLKFLKEYIAPVTERLYPGYYPKAQAIMNFVVRYHPDEQPFLRPHHDSSTFTINIALNRKNIDYEGGGCRFLRYNCNVESPRKGWSFMHPGRLTHYHEGLPTTKGTRYIMVSFVDP

SEQ ID NO: 40 - protein, 730 aa, Caenorhabditis elegans
  SIGNAL (1)..(16): signal sequence
MRVLPFLLPLIPVLLATTITDLPELVVVTVATENTDGLKRLLESAKAFDINIEVLGLGEKWNGGDTRIEQGGGQKIRILSDWIEKYKDASDTMIMFVDAYDVVFNADSTTILRKFFEHYSEKRLLFGAEPFCWPDQSLAPEYPIVEFGKRFLNSGLFMGYGPEMHKILKLKSVEDKDDDQLYYTMIYLDEKLRKELNMDLDSMSKIFQNLNGVIEDVELQFKEDGTPEAYNAAYNTKPLIVHGNGPSKSHLNYLGNYLGNRWNSQLGCRTCGLEVKESEEVPLIALNLFISKPIPFIEEVLQKIAEFDYPKEKIALYIYNNQPFSIKNIQDFLQKHGKSYYTKRVINGVTEIGDREARNEAIEWNKARNVEFAFLMDGDAYFSEPKVIKDLIQYSKTYDVGIIAPMIGQPGKLFTNFWGAIAANGYYARSEDYMAIVKGNRVGYWNVPFITSAVLFNKEKLEAMKDAYSYNKNLDPDMSMCKFARDNGHFLYIDNEKYYGFLIVSDEYAETVTEGKWHPEMWQIFENRELWEARYIHPGYHKIMEPEHVVDQACPDVYDFPLMSERFCEELIEEMEGFGRWSDGSNNDKRLAGGYENVPTRDIHMNQVGFERQWLYFMDTYVRPVQEKTFIGYYHQPVESNMMFVVRYKPEEQPSLRPHHDASTFSIDIALNKKGRDYEGGGVRYIRYNCTVPADEVGYAMMFPGRLTHLHEGLATTKGTRYIMVSFINP

SEQ ID NO: 41 - protein, 721 aa, Drosophila melanogaster
MRIQQSALLLLLLAVTSQGDAESNWNDKIKVFTVATEPTDGYTRYIRSARVYDIEVTTLGLGEEWKGGDMQKPGGGFKLNLLREAIAPYKNEPETIILFTDSYDVIITTTLDEIFEKFKESGAKILFSAEKYCWPDKSLANDYPEVEGKASRFLNSGAFIGYAPQVFALLVDPIEDTADDQLYFTKIFLDETKRAKLGLKLDVQSRLFQNLHGAKNDVKLKVDLESNQGVLQNVDFMTTPSIIHGNGLSKVDLNAYGNYLARTFNGVCLLCQENLLDLEETNLPVISLALMVTQPVPFFDQFLEGIESLNYPKEKLHLLIYSNVAFHDDDIKSFVNKHAKEYATAKFALSTDELDERQGRQLALDKARLHQSDYIFFVDADAHIDDGEVLRELLRLNKQFVAPIFSKHKELWSNFWGALSEGGYYARSHDYVDIVKRELIGMFNVPHVTSIYLVKKTAFDAISFKHKEFDPDMAMCESLRNAGIFMYASNLRIFGHLVNADDFNTTVTRPDFYTLFSNEIDWTEKYIHPNYSLQLNESNKIQQPCPDVYWFQIVSDAFCDDLVAIMEAHNGWSDGSNNDNRLEGGYEAVPTRDIHMKQVGLERLYLKFLQMFVRPLQERAFTGYFHNPPRALMNFMVRYRPDEQPSLRPHHDSSTYTINIAMNRAGIDYQGGGCRFIRYNCSVTDTKKGWMLMHPGRLTHYHEGLLVTNGTRYIMISFIDP

SEQ ID NO: 42 - protein, 707 aa, Aedes aegypti
MFRKLEYNISQKPPLVFTVASNATEGYLRYIRSAKYYGIEVSTLGLGKPWLGGDMTRLGGGYKINLLRDALKPYKADDDRIVLFTDSYDVLFLASMEKIIEKFRTFDASILFGSEGFCWPEEDLKSKYPVLEGRGTRFLNSGLFMGYASKVYRMLKTPVKDTDDDQLYYTKAYLDEKQRNELKIKLDHTAVLFQNLNGVEEQVVLALDENGKEAFLKNTEYSTVPYIVHGNGPSKLVLNGYANYLAGAFVDGECKTINEDLIQLDEENLPTVMLALFIEKATPFIEEWFEGIAKINYPSKKMDLFIHNNVDYHKPTIDDFIEKYSSSYRSFRMVDYTDDYEELAGRSLAVDQCLKKQCDYLFVVDADGHIDDSDIIRKLIVQNKSIISPMLNRPEKVWSNFWGALSSQGFYARSSDYMDIVGRKILGQWNVPYISTIYLVKASVLPLVSYELQGTDPDMALCWHMRAKGIFMHVINAEQYGHLIDSDYYDTTKTHPDFYQLFNNKHDWEQKYISPEYYKQLEKDYVQIQPCPDVYWFAIASELFCDHLKEIVEAFGKWSDGTHTDKRLQGGYEAVPTRDIHMNQVGLEQVWLKFLQLYVKPLQEKVFIGYYHDPPRSLMNFVVRYRPDEQPSLRPHHDSSTYTINIALNRAGIDYEGGGCHFLRYNCSVTDTRKGWMLMHPGRLTHFHEGLRTNSGTRYIMISFVDP

SEQ ID NO: 43 - protein, 895 aa, Acanthamoeba polyphaga minivirus
MISRTYVINLARRPDKKDRILAEFLKLKEKGVELNCVIFEAVDGNNPEHLSRFNFKIPNWTDLNSGKPMTNGEVGCALSHWSVWKDVVDCVENGTLDKDCRILVLEDDVVFLDNFMERYQTYTSEITYNCDLLYLHRKPLNPYTETKISTHIVKPNKSYWACAYVITYQCAKKFMNANYLENLIPSDEFIPIMHGCNVYGFEKLFSNCEKIDCYAVQPSLVKLTSNAFNDSETFHSGSYVPSNKFNFDTDKQFRIVYIGPTKGNSFHRFTEYCKLYLLPYKVIDEKETNDFVSLRSELQSLSEQDLNTTLMLVVSVNHNDFCNTIPCAPTNEFIDKYKQLTTDTNSIVSAVQNGTNKTMFIGWANKISEFINHYHQKLTESNAETDINLANLLLISSISSDFNCVVEDVEGNLFQLINEESDIVFSTTTSRVNNKLGKTPSVLYANSDSSVIVLNKVENYTGYGWNEYYGYHVYPVKFDVLPKIYLSIRIVKNANVTKIAETLDYPKELITVSISRSEHDSFYQADIQKFLLSGADYYFYISGDCIITRPTILKELLELNKDFVGPLMRKGTESWTNYWGDIDPSNGYYKRSFDYFDIIGRDRVGCWNVPYLASVYLIKKSVIEQVPNLFTENSHMWNGSNIDMRLCHNLRKNNVFMYLSNLRPYGHIDDSINLEVLSGVPTEVTLYDLPTRKEEWEKKYLHPEFLSHLQNFKDFDYTEICNDVYSFPLFTPAFCKEVIEVMDKANLWSKGGDSYFDPRIGGVESYPTQDTQLYEVGLDKQWHYVVFNYVAPFVRHLYNNYKTKDINLAFVVKYDMERQSELAPHHDSSTYTLNIALNEYGKEYTAGGCEFIRHKFIWQGQKVGYATIHAGKLLAYHRALPITSGKRYILVSFVN

SEQ ID NO: 44 - protein, 688 aa, Brugia malayi
MTGMTLWVLTLSTVLMYGTVTMEKISGMPELLVVTVATEETDGLRRLKRTADINDVGLEVFGMGEQWRGGDVRVDKGGGQKIRILRKSLEKYKDRNDLIILFVDAYDVILLGNEEQILRNFFTFFDGFRLVFSSEPFCWPNRSLAPKYPLVNFGYRYLNSGVFMGFAPEIWNLISYKDVEDNDDDQLYYTRLYLDEQIRMSLKMTLDSMSILFQNLNGASNDVKLEMSDERSGTYFDLEKIELPRLFLSVIISKPIPFIREFFENIKSLVYADEKIDLYVYCNQNFLEKETNGFVEDVKGRYRSLLYDGSTTELGEREARAFSLKQSLALGDDYLIMIDGDVHLNNSEALLLMIHRVKEKDSEILAPLVGQPHKLFTNFWGAISSNGYYARSENYLDIIDYKEVGIWNVPFISSILIIAKEKLTSLSNAYYYNDKLDPDMSFCSFARDKGHFLYLDNSHYYGFLVVSEDVESSKVHPDMYEIFNNKELWEKRYIHPNYFAALNGSIQILEICQDVYDFPLMSERFCAELIEECEYYGKWSDGKHKDERLVGGYENVPTRDIHMNQIGFERHWLYMLDEYVRPIQEKLFIGYYKQPVESVMMFVVRYKPEEQASLRPHHDASTYSIDIALNKRGVDYEGGGVRFLRYNCTFDADTVGHSMIFPGRLTHLHEGLETTQGTRYIAVSFINP

SEQ ID NO: 45 - protein, 702 aa, Ornithorhynchus anatinus
MAAPRPAFPSLLLLLLLLLPGLPAARAGDGPPAGERVNPEKLLVMTAATEETEGYKRFLRTARHFNYTVRTLGLGEEWRGGDVARTVGGGQKVRWLKQEMEKHADREDLVILFVDSYDVLLAGSPLELLWKFVQSGSRLLFSAEGFCWPEWSLADSYPPLSAGNGKRFLNSGGFIGFAPTVHRLVRQWKYKDDDDDQLFYTRLYLDPGLREKHGLALDHKSRIFQNLNGALDEVVLKFEKNRVRVRNVAYDTLPVVIHGNGPTKLQLNYLGNYVPNAWTYEGGCGFCAQDRRNLTGDSELPRVLLGLFVEQPTPFLPQFLQRLLLLDYPSSRLSLFLHNSEVYHEAHVEALWEQLRTRFSTVQLVGPEEALTQGEARDMAMDSCRQDPSCDFYFSLDADAVLTNPRTLLSLIEEDRKVVAPMLSRHGKLWSNFWGALSPEEYYARSEDYVELVQRKRVGLWNVPYVAQAYLVRGETLRSELPQRGVFTLEETDPDMSFCKSLRDKGIFLHLSNQEEFGRLVSTARYDTDHLHPDLWQIFDNPLDWREKYIHPNYSLALEGEGVEQPCPDVYWFPVLSDRMCDELVEEMENFGQWSGGRHEDTRLAGGYENVPTVDIHMNQVGYEKEWLKVLSEYIAPMTESLFPGYHTKADRTEGTNSSWASDHTSSPANISRSDDPPPPAEPDLGGPGAPPPAKKDGGRGI

SEQ ID NO: 46 - protein, 726 aa, Nasonia vitripennis
MKRWTCVLLAVLACVAAEETDDALVFTVATNETEGFRRYLRSTEVNGFGDNVRVLGLGQAWRGGEIKLYAGGGQKVNLLKEAIEEIKDDPDQIVLFTDSYDVIFLSSLEKISRKFKEWDDARVIFSAEEYCWPLKSLASEYPQVKRGKRFLNSGGFIGYAPDIYAILTSAEIKDDDDDQLFYTKVYLNSELREKHKIKLDHKSEIFQNLNGAIHDIELRFKGNEAYVQNTAYNTVPLIIHGNGFSKLLLNSLGNYVAQAWSPEEGCLSCWDRTIELDVKNAEAYPKILIAIFIEKPTPFLEEFLNKIKDQRYPKEKLHFFIRNNVPYHEKLIDEFVEKHGDEYQSVKQIKPEDEIAEAAARNLAMNHCLSVKCSGYFSIDSESHLDNVNTLELLVEQQRGIVAPLLVRPFKAWSNFWGAITDDGFYARSSDYMDIIHHERRGLWNVPFVSSCYLINATLLENEATRPSYAEADLDAEMAFAYANRRRDIFMYVNNRLDFGHLVNPETFNISLTNPDMYQMFDNKLDWEKRYIHVNYSDNFLPENKPVQPCPDVYWFPIVTERFNKDFVEIMEAYGKWSDGSNYDPRLSNGYENVPTRDIHMNQVGLESQWLFFLRNYVKPLQELVFLGYFHDPPRSLMNFVVRYRPDEQPSLKPHHDSSTYTINIALNKVGVDYEGGGCRFIRYNCSVTDTKPGWMLMHPGRLTHYHEGLKVTKGTRYIMISFVDP

SEQ ID NO: 47 - protein, 714 aa, Pan troglodytes
MGQASVRHDCYPDNLLVLTVATKETEGFRRFKRSAQFFNYKIQALGLGEDWNVDKGTSAGGGQKVRLLKKALEKHADKEDLVILFTDSYDVLFASGPRELLKKFRQARSQVVFSAEELIYPDRRLETKYPVVSDGKRFLGSGGFIGYAPNLSKLVAEWEGQDSDSDQLFYTKIFLDPEKREQINITLDHRCRIFQNLDGALDEVVLKFEMGHVRARNLAYDTLPVLIHGNGPNKLQLNYLGNYIPRFWTFETGCTVCDEGLRSLKGIGDEALPTVLVGVFIEQPTPFVSLFFQRLLRLHYPQKHMRLFIHNHEQHHKAQVEEFLAEHGSEYQSVKLVGPEVRMANADARNMGADLCRQDRSCTYYFSVDADVALTEPNSLRLLIQQNKNVIAPLMTRHGRLWSNFWGALSADGYYARSEDYVDIVQGRRVGVWNVPYISNIYLIKGSALRGELQSPDLFHHSKLDPDMAFCANVRQQDVFMFLTNRHTLGHLLSLDSYRTTHLHNDLWEVFSNPEDWKEKYIHQNYTKALAGKLVETPCPDVYWFPIFTEVACDELVEEMEHFGQWSLGNNKDNRIQGGYENVPTIDIHMNQIGFEREWHKFLLEYIAPMTEKLYPGYYTRAQFDLAFVVRYKPDEQPSLMPHHDASTFTINIALNRVGVDYEGGGCRFLRYNCSVRAPRKGWTLMHPGRLTHYHEGLPTTRGTRYIAVSFVDP

SEQ ID NO: 48 - protein, 729 aa, Nemostella vectensis
MSVKALISSCVFLLASLSYLVNADNGFSRDPKELELLVLTVATEETDGYTRFMRSCSHYDVPVRVIGMNTSWKGGNVRTDPGGAHKINLLKDAVAEYKDKKNLVLMFSDSYDAIFLARAEAFIKKFLEFKAHVVFSAEGFCWPDRWLVDKYPEVGHGKRYLCSGGFIGYAPVFHQIINEKPVKDEDDDQLFYTNIYLDKEKRDKFNMKLDHKAEIFMNLNGAEEEVQLKFEGEKVWLYNKVYSTTPLWVHGNGPSKVHLNYIGNYLPAMWNKEKGCLVCNEDTIKLPEKESDYPKVMMAIFISRPTPFVPEFFKRIEALDYPKKKIALYIHNLMDGHTKEVNEWLTEEIRGLYHSVTYQGPGTFEAAARNKAVYSGSDYLFVVDANVVYTNKKSLKLLIEQNRPLLVPKMSKHAKLWSNFWGTIGDDGYYARAEDYIDIVEYRRVGIWNSAYVTGSYLIQKDVLPKLKHAYSYGNLEPDLSFSKYLRDNGIFMYVTNMHYFGRLKETDTVTTNHLHNDLWQIFDNQIDWEERYLHPNYSQNLNKSIPLKMPCNDVFWFPLMSETWATHMIEEMEHYGKWSGGKHEPQDARLNGGYENVPTVDIHMNQVGWEREWLHLLKTYIVPVNTRIFPGYYSEGRAIMNFVVKYTPSGQYYLRPHHDSSTYTINIGLNKPGIHYGGGGSRFIRQDCAVTDTQVGWALMHPGRLTHYHEGLPTTWGTRYIMVCFVDP

SEQ ID NO: 49 - protein, 743 aa, Equus caballus
MASSGAGPRLLLLLLLLLLLLPPPPAASASDRPRGSDPVNPEKLLVITVATAETEGYRRFLRSAEFFNYTVRTLGLGEDWRGGDVARTVGGGQKVRWLKKEMEKYADREDMVIMFVDSYDVILAGSPSELLKKFVQSGSRLLFSAESFCWPEWGLAEQYPEVGTGKRFLNSGGFIGFAPTIHQIVRQWKYKDDDDDQLFYTRLYLDPGLREKLSLNLDHKSRIFQNLNGALDEVVLKFDRNRVRIRNVAYDTLPVVVHGNGPTKLQLNYLGNYVPKGWTPEGGCGYCDLDRRTLPGGQPPPRVLLAVFVEQPTPFLPRFLQRLLLLDYPPDRVALFLHNNEVYHEPHIADSWPQLQDHFSAVKLVGPEEALTPGEARDMAMDSCRQDPKCEFYFSLDADAVITNPQTLRILIEENRKVIAPMLSRHGKLWSNFWGALSPDEYYARSEDYVELVQRKRVGVWNVPYISQAYVIRGETLRTELPQKEVFSSSDTDPDMAFCKSLRDQGIFLHLSNRHEFGRLLATSRYDTDHLHPDLWQIFDNPLDWKEQYIHENYSRALEGKGLVEQPCPDVYWFPLLSDQMCDELVEEMEHYGQWSGGRHEDSRLAGGYENVPTVDIHMKQVGFEDQWLQLLRTYVGPMTESLFPGYHTKTRAVMNFVVRYRPDEQPSLRPHHDSSTFTLNVALNHKGLDYEGGGCRFLRYDCVVSSPRKGWGLLHPGRLTHYHEGLPTTRGTRYIMVSFVDP

SEQ ID NO: 50 - protein, 710 aa, Anopheles gambiae
MALFSHFPRIPASTKEPLIFTVASNATEGYVRYLRSAKHYDLTVTTLGMGKPWLGGNMKSVGGGYKINLLREALKPYRADKDRLVLFTDSYDVLFLAPWAKIQEKFASFEASILFGAEGFCWPDESLKSAYPPLEGRGMRYLNSGLFMGYADKLYKLLKTPVKDAEDDQLYYTKAYLDEELRQELNIKLDHMATLFQNLNGVEEQVVLSLEPSEKEATLANSEYNTKPAIVHGNGPSKLTLNSYANYLAGAFVDGECQTVKEGRLTLSGGELPLVTMALFVEKPTPFLEEWFGTIAKLNYPADRLDVLVHSNVAYHAGTVKAFLDAQEGRYRSLKVIEHDGDFTETAARNFATKHCELRGCDYLFVVDSEGHLDDPNVLRALIEANRNVIAPVLTRPEKVWSNFWGALSGQGFYARSNDYMDIVGRKLLGLWNVPFVSIVYLVKRAVLPEVSYELQETDPDMALCWHFRSKGIFMHVINVEQYGHLIDTEYFDMTRTHPDFYQLFNNRHDWEQRYLAPGYKQQLEADFVPQQPCPDVYWFAIGSDRFCDDLREIVEAFGEWSDGSHSDKRLQGGYEAVPTRDIHMNQVGLEQLWLKFLQLYVRPLQEKVFIGYFHDPPRSLMNFVVRYRPDEQPSLRPHHDSSTYTINIALNTAGVDYEGGGCRFLRYNCSVTDTRKGWMLLHPGRLTHFHEGLLTTKGTRYIMISFVDP

SEQ ID NO: 51 - protein, 733 aa, Caenorhabditis briggsae
MRVLPLLLPLLIIPVILAVSITDLPELVVVTVATENTDGLKRLLESAKAFDINIEVLGLGEKWNGGDTRVEKGGGQKIRILSKWIEKYKDASDTIIMFVDAYDVVFNADSKNILQKFLEHYPGKQLLFGAEPFCWPDQTLAPDYPIVEFGKRFLNSGLFMGYGPQVHKILTLKSVEDKDDDQLYYTMIYLDEKLRKELNMDLDSMSKIFQNLNGVIEDVELQFKEDGTPEAYNAAYNTKPLIVHGNGPSKSHLNYLGNYLGNRWNSQLGCRTCDQEGAKEQTEFPLIGLNLFISKPVPFIEEVLQKVSEFDYPKNRIALYIYNNQPFSIKNIQDFLKDHGKSYYTKRIINGVTEIGERQARNEAIDWCKQRDTEFAFFMDGDAYFTEPTVIKDLIHYSKSYDVGIISPMVGQPGKLFTNFWGAIAANGYYARSEDYMAIVKGNRVGYWNVPFVTSALLMSKEKLGAMSGAYTYNKNLDPDMSLCQFARDNGHFMYINNEKYFGYLIVSDEFSETVTEGKWHPEMWQIFENRELWEARYIHPGYHKIMEPDHIIDQACPDVYDYPLMSERFCEELIEEMEGFGRWSDGSNNDKRLAGGYENVPTRDIHMNQVGFERQWLYFLDTYVRPVQEKTFIGYYHQPVESNMMFVVRYKPEEQASLRPHHDASTFSIDIALNKKGRDYEGGGVRYVRYNCTVEADEVGYAMMFPGRLTHLHEGLATTKGTRYIMVSFINP

SEQ ID NO: 52 - protein, 695 aa, Drosophila pseudoobscura
DKVEVFTVATEPTDGYARYIRSARIYDVKVTTLGLGEHWKGGDMQHPGGGFKVNLLRKAVAPLKDEQDTIVLFTDSYDVIITAKLEEIVELFKESKAKLLFSAEKFCWPDSSLTDAYPEVEGNASRFLNSGAFIGYAPQVNALLEEAIDDMDDDQLYYTKVFLDEARRAKLGMKLDTQSRLFQNLHGAKNDVKLKVDIESNQGILQNVNFLTTPAIVHGNGLSKVDLNAYGNYLAKTFNGICTVCQEYLLELDEQHLPVISLSVIVPMAVPFFDQFLEGIEKINYPKQNLHLLIYSNVELHDADIKSFVNKHGEKYASAKYTLSTDNLDERQGRQLAFDQAKLRKSDYIFFIDGDAHIDDGEVLRELLKLNKQFVAPLFAKYHELWSNFWGALSEGGFYARSHDYVDIVKRDLIGIFNVPHVTSIYLVRSSVFDVLSFQHSEYDADMAMCESLRKAGVFMFISNQRYFGHLVNADNFDTKVARPDFYTLFSNRYDWTEKYIHPNYSEQLNASTVIEQPCPDVYWMAIVTDAFCDDLVAIMENHGTWSDGSNNDNRLEGGYEAVPTRDIHMKQVGLEVLYLKFLELFVRPLQERVFTGYYHNPPRALMNFMVRYRPDEQPSLRPHHDASTYTINIAMNQVDTDYEGGGCRFLRYNCSVTETKKGWMLMHPGRLTHYHEGLLVTKGTRYIMISFIDP

SEQ ID NO: 53 - DNA, 2217 bp, Homo sapiens
atgacctcctcggggcctggaccccggttcctgctgctgctgccgctgctgctgccccctgcggcctcagcctccgaccggccccggggccgagacccggtcaacccagagaagctgctggtgatcactgtggccacagctgaaaccgaggggtacctgcgtttcctgcgctctgcggagttcttcaactacactgtgcggaccctgggcctgggagaggagtggcgagggggtgatgtggctcgaacagttggtggaggacagaaggtccggtggttaaagaaggaaatggagaaatacgctgaccgggaggatatgatcatcatgtttgtggatagctacgacgtgattctggccggcagccccacagagctgctgaagaagttcgtccagagtggcagccgcctgctcttctctgcagagagcttctgctggcccgagtgggggctggcggagcagtaccctgaggtgggcacggggaagcgcttcctcaattctggtggattcatcggttttgccaccaccatccaccaaatcgtgcgccagtggaagtacaaggatgatgacgacgaccagctgttctacacacggctctacctggacccaggactgagggagaaactcagccttaatctggatcataagtctcggatctttcagaacctcaacggggctttagatgaagtggttttaaagtttgatcggaaccgtgtgcgtatccggaacgtggcctacgacacgctccccattgtggtccatggaaacggtcccactaagctgcagctcaactacctgggaaactacgtccccaatggctggactcctgagggaggctgtggcttctgcaaccaggaccggaggacactcccgggggggcagcctcccccccgggtgtttctggccgtgtttgtggaacagcctactccgtttctgccccgcttcctgcagcggctgctactcctggactatccccccgacagggtcacccttttcctgcacaacaacgaggtcttccatgaaccccacatcgctgactcctggccgcagctccaggaccacttctcagctgtgaagctcgtggggccggaggaggctctgagcccaggcgaggccagggacatggccatggacctgtgtcggcaggaccccgagtgtgagttctacttcagcctggacgccgacgctgtcctcaccaacctgcagaccctgcgtatcctcattgaggagaacaggaaggtgatcgcccccatgctgtcccgccacggcaagctgtggtccaacttctggggcgccctgagccccgatgagtactacgcccgctccgaggactacgtggagctggtgcagcggaagcgagtgggtgtgtggaatgtaccatacatctcccaggcctatgtgatccggggtgataccctgcggatggagctgccccagagggatgtgttctcgggcagtgacacagacccggacatggccttctgtaagagctttcgagacaagggcatcttcctccatctgagcaatcagcatgaatttggccggctcctggccacttccagatacgacacggagcacctgcaccccgacctctggcagatcttcgacaaccccgtcgactggaaggagcagtacatccacgagaactacagccgggccctggaaggggaaggaatcgtggagcagccatgcccggacgtgtactggttcccactgctgtcagaacaaatgtgtgatgagctggtggcagagatggagcactacggccagtggtcaggcggccggcatgaggattcaaggctggctggaggctacgagaatgtgcccaccgtggacatccacatgaagcaggtggggtacgaggaccagtggctgcagctgctgcggacgtatgtgggccccatgaccgagagcctgtttcccggttaccacaccaaggcgcgggcggtgatgaactttgtggttcgctaccggccagacgagcagccgtctctgcggccacaccacgactcatccaccttcaccctcaacgttgccctcaaccacaagggcctggactatgagggaggtggctgccgcttcctgcgctacgactgtgtgatctcctccccgaggaagggctgggcactcctgcaccccggccgcctcacccactaccacgaggggctgccaacgacctggggcacacgctacatcatggtgtcctttgtcgacccctga

SEQ ID NO: 54 - DNA, 24 bp, Artificial
  oligonucleotide; forward primer
tggttgcaaagctgaaacttaaag

SEQ ID NO: 55 - DNA, 20 bp, Artificial
  oligonucleotide; reverse primer
agtcaaattaagccgcaggc

SEQ ID NO: 56 - DNA, 21 bp, Artificial
  oligonucleotide; probe
cctggtggtgcccttccgtca

(19) United States
(12) Patent Application Publication
(10) Pub. No.: US 2010/0124548 A1
(43) Pub. Date: May 20, 2010

(54) Pharmaceutical product

(75) Inventors: Raili Myllylä, Oulu (FI); Heli Ruoisalainen, Oulu (FI)

Correspondence Address: DODDS & ASSOCIATES, 1707 N STREET NW, WASHINGTON, DC, 20036, US

(73) Assignee: University of Oulu, Oulun Yliopisto (FI)

(21) Appl. No.: 12/589,882

(22) Filed: Oct. 29, 2009

Related U.S. Application Data
(60) Provisional application No. 61/197,642, filed on Oct. 29, 2008.

(51) Int. Cl. A61K 38/54; A61K 38/46; A61K 38/45; C12N 9/14; C07H 21/04; C12P 21/06; A61K 31/7088; C12Q 1/48; C12Q 1/34

(52) U.S. Cl. 424/94.2; 424/94.6; 424/94.5; 435/195; 536/23.2; 435/69.1; 514/44 R; 435/15; 435/18

(57) ABSTRACT

The present invention relates to methods and products for the treatment of any disorder or condition, which is associated with abnormal amount of non-collagenous protein, or abnormal oligomerization or dysfunction of non-collagenous protein, such as adiponectin in the blood circulation and/or tissue of a patient. The treatment comprises that functional form of the non-collagenous protein is adjusted in the blood circulation and/or tissue of the patient substantially to the level it is in the blood circulation and/or tissue of a healthy person, by using lysyl hydroxylase and/or glycosyl-transferase activities of LH3 or other lysyl hydroxylase to modify the non-collagenous protein to HMW or other functional form.

### PRIORITY

This application claims priority of U.S. provisional application No. 61/197,642 filed on Oct. 29, 2008.

### SEQUENCE LISTING

This application contains sequence data provided on a computer readable diskette and as a paper version. The paper version of the sequence data is identical to the data provided on the diskette.

### FIELD OF THE INVENTION

The present invention relates to new products and methods, which can be used in treating disorders or conditions, which are associated with abnormal amount of non-collagenous proteins, or abnormal oligomerization or dysfunction of non-collagenous proteins in the body of a patient. In particular, the present invention relates to disorders or conditions, where the non-collagenous protein is adiponectin.

### BACKGROUND ART

According to the World Health Organization there were at least 171 million people world wide who suffer from diabetes in year 2000, and the estimate for year 2030 is 366 million people. The incidence of the disease is thus increasing rapidly. The increase in incidence seems to follow the trend of urbanization and “Western style” diet. However, the mechanism(s) of the disease is poorly known at present. With regard to type 2 diabetes, fat concentrated around the waist in relation to abdominal organs is known to predispose individuals for insulin resistance. Abdominal fat is known to be especially active hormonally, secreting a group of hormones called adipokines that are shown to impair glucose tolerance. A patient having diabetes has increased risk for heart attack or stroke.

Adiponectin is an adipokine that is secreted by adipocytes. Adiponectin has been shown to have antidiabetic, antiatherogenic, and anti-inflammatory properties. It exists in the blood circulation as a multimeric protein. Serum adiponectin consists of trimer, hexamer, and larger high-molecular weight (HMW) multimers. HMW multimers are the most bioactive species and the ratio of HMW to total adiponectin, rather than the total levels, provides the closest correlation with measures of insulin sensitivity. Reduced serum levels of HMW adiponectin are also associated with coronary artery disease. The vascular protective effects of adiponectin were shown to be restricted to the HMW component (Richards et al., 2006 and the cited references). Also Nedvídková et al 2005, Tilg & Moschen 2006 report that the total adiponectin level and the level of HMW adiponectin are lowered in insulin resistant states as obesity, type 2 diabetes and coronary artery disease.

Wang et al. 2006 have shown that the three oligomeric forms of adiponectin, trimeric, hexameric, and high molecular weight (HMW) oligomeric complexes, are differentially glycosylated, when adiponectin was produced from a mammalian cell, with the HMW oligomer having the highest carbohydrate content. Richards et al. 2006 have reported that mutation of modified lysines in the collagenous domain prevented formation of HMW multimers, and pharmacological inhibitor of prolyl- and lysyl-hydroxylases, 2,2′-dipyridyl, inhibited formation of hexamers and HMW multimers.

It is thus known that the oligomerization state of adiponectin is dependent on the level of lysine hydroxylation and glycosylation in the collagenous domain of adiponectin. However, it is not known how the oligomerization of adiponectin is regulated. Wang et al. 2008 remarks that the biosynthesis and secretion of adiponectin in adipocytes is a complex process that involves several types of posttranslational modifications (PTM). The secretion of adiponectin oligomers, especially HMW adiponectin, is tightly controlled by a pair of endoplasmic reticulum(ER)-resident proteins Erp44 and Ero1-Lα, whereas the circulating concentrations of HMW adiponectin are selectively increased by PPARγ agonist through up-regulation of Ero1-Lα expression.

There are nearly 20 proteins that are not members of the collagen family, but all these proteins have a short, at least 6 Xaa-Yaa-Gly repeats long (Xaa and Yaa any amino acids) collagenous triple-helical domain in their structure. Little is known about the role of the post-translational lysine modifications for the function of these proteins. At least adiponectin, mannan-binding lectin, C1q subcomponent of complement activation and surfactant proteins D and acetylcolinesterase are known to have Glc-Gal-Hyl residues in their collagenous domain, but it is not known which enzyme is responsible for the catalysis of these posttranslational lysine modifications.

In adiponectin and mannan-binding lectin the glycosylated hydroxylysines have been reported to have a role in the formation and secretion of higher oligomeric forms (Heise et al. 2000; Wang et al. 2002a; Richards et al. 2006; Wang et al. 2006).

Studies on peptides of collagenous Xaa-Yaa-Gly sequences have demonstrated a marked effect of chain length, in that of Kₘ decreases with increasing chain length, when Kₘ is expressed as molar concentrations of triplets or of the peptide. This holds true for all post-translational enzymes of collagen biosynthesis, i.e. prolyl-4-hydroxylase, lysyl hydroxylase, galactosyltransferase and glucosyltransferase. There is many thousands fold difference in Kₘ value, if compared short sequence with long sequence (procollagen) (Kivirikko and Myllyla, 1979, 1980). It is known that long collagenous proteins, which have Xaa-Yaa-Gly- repeats of many hundreds, are good substrates for lysine modifying enzymes, but short proteins, such as non-collagenous proteins having only short, 6 to 40 Xaa-Yaa-Gly repeats (for example adiponectin has 22 repeats of Xaa-Yaa-Gly) are less suitable substrates for lysine modifying enzymes. Although it is known that human and other mammals have lysyl hydroxylase 3 enzyme, that has capability of both hydroxylation and glycosylation of lysine residues (WO 0192505) in collagenous proteins, its role and significance in the hydroxylation and glycosylation of non-collagenous protein is completely unknown. In regard to adiponectin, the regulation of its posttranslational modifications and oligomer composition is complex and seems to occur at multiple levels through multiple mechanisms.

There is thus a clear need for finding a method or a factor for increasing the amount or activity of adiponectin and in particular HMW oligomer of adiponectin in the human body.

In addition to disorders or conditions, which are related to the amount and oligomerization of adiponectin, there are many other non-collagenous protein related diseases, the diagnosis and treatment of which needs to be developed.

### SUMMARY OF THE INVENTION

The present invention eliminates at least some problems of the prior art.

In particular, the present invention provides methods and products for the treatment of disorders or conditions, which are associated with abnormal amount or abnormal oligomerization or dysfunction of specific non-collagenous proteins.

More specifically, the present invention provides methods and products for the treatment of disorders or conditions, which are associated with abnormal amount, abnormal oligomerization or dysfunction of specific non-collagenous proteins in the blood circulation and/or tissue of a patient.

In particular, the present invention provides methods and products for the treatment of disorders or conditions, which are associated with abnormal amount of adiponectin or abnormal oligomerization or dysfunction of adiponectin in the blood circulation and/or tissue of a patient.

The present invention is based on the surprising finding that the absence of lysyl hydroxylase activity of lysyl hydroxylase 3 (LH3) reduces the amount of total adiponectin and high molecular weight (HMW) form of adiponectin in the serum of mice. This result indicates that LH3 hydroxylates and further glycosylates hydroxylysine residues in adiponectin and thus affects the oligomerization of adiponectin.

Adiponectin is a non-collagenous protein which has a signal peptide, a variable N-terminal domain, followed by a collagenous domain comprising 22 Gly-Xaa-Yaa repeats and a C-terminal globular domain (Wang et al., 2008). The glucosylgalactosylhydroxylysine residues locate on the surface of the collagenous triple helix, thereby being able to participate in intra- and intermolecular interactions and thus affect the structure and function of the protein. Since other non-collagenous proteins have similar lysine modifications, the present invention can be applied also to other non-collagenous proteins.

According to the present invention, non-collagenous protein, and/or in particular HMW or other functional form of the non-collagenous protein, is adjusted in the blood circulation and/or tissue of the patient substantially to the level it is in the blood circulation and/or tissue of a healthy person, by using lysyl hydroxylase and/or glycosyltransferase activity/activities to modify the non-collagenous protein to HMW or other functional form.

In particular, the present invention comprises that lysyl hydroxylase 3 (LH3) or other lysyl hydroxylase (LH) having lysyl hydroxylase and glycosyltransferase (GT and GGT) activities or a fragment or modified form of these having at least one of these activities, is used to modify the non-collagenous protein in the body of a patient or in a cell or tissue culture producing the non-collagenous protein.

More specifically, the method according to the present invention is mainly characterized by what is stated in the characterizing part of claims 1 and 9.

Lysyl hydroxylase 3 (LH3) or other lysyl hydroxylase (LH) or a nucleic acid sequence encoding LH3 or LH are mainly characterized by what is stated in the characterizing part of claims 20 and 21.

A method for producing non-collagenous protein in HMW or other functional form is mainly characterized by what is stated in the characterizing part of claim 22.

A method for preparing a medicament for the treatment of a disorder or condition, which is associated with abnormal amount of non-collagenous protein, or abnormal oligomerization or dysfunction of non-collagenous protein in the blood circulation and/or tissue of a patient, is mainly characterized by what is stated in the characterizing part of claim 30.

Pharmaceutical compositions are mainly characterized by what is stated in the characterizing part of claims 31 to 34.

A method for diagnosing a disorder or condition associated with abnormal amount of non-collagenous protein, or abnormal oligomerization or dysfunction of non-collagenous protein in the blood circulation and/or tissue of a patient is mainly characterized by what is stated in the characterizing part of claim 35.

The present invention can be applied to diseases or conditions, which are associated with abnormal amount, abnormal oligomerization or dysfunction of specific non-collagenous proteins in the blood circulation and/or tissue of a patient. The non-collagenous protein is preferably selected from the group of proteins that
comprise glucosylgalactosylhydroxylysine, such as adiponectin, mannan-binding lectin, C1q subcomponent of complement activation, surfactant protein D, collectin-43; or
comprise at least hydroxylysine, such as surfactant protein A, collagenous tail (collagen Q) of asetylcholineesterase or burylcholinesterase, conglutinin, collectin-46; or
comprise lysine in the Yaa position in the Xaa-Yaa-Gly repeat, such as collectin liver 1 (CL-L1), collectin placenta 1 (CL-P1), collectin kidney 1 (CL-K1), macrophage receptor MARCO, macrophage scavenger receptor type I, macrophage scavenger receptor type II, C1q, tumor necrosis factor related protein (C1qTNF) 1, 2, 3 (also called CORS-26 or cartonectin), 5, 6, 7, 8, otolin-1, adipoQ-like 1 (AQL1), adipoQ-like 2 (AQL2), gliacolin 1, gliacolin 2, collagen triple helix repeat containing 1, gliomedin, CRF 1 and CRF 2.

More preferably the present invention can be applied to diseases or conditions which are associated with abnormal oligomerization or dysfunction of adiponectin and/or mannan-binding lectin or other non-collagenous proteins having structural and/or functional similarities with these proteins.

According to one preferred embodiment of the invention the treatment comprises that lysyl hydroxylase or glycosyltransferase activity or both activities of LH3 are adjusted in the blood circulation and/or tissue of the patient substantially to the level they are in the blood circulation and/or tissue of a healthy person.

Within the scope of the present invention are also disorders or conditions where abnormal amount of non-collagenous protein, or abnormal oligomerization or dysfunction of non-collagenous protein in the blood circulation and/or tissue of a patient can not be shown, but the treatment comprises that lysyl hydroxylase and/or glycosyltransferase activity or activities of LH3 or non-collagenous protein, preferably in the HMW oligomeric form or other functional form is/are increased or adjusted to more appropriate level in the blood circulation and/or tissue of the patient, by using lysyl hydroxylase and/or glycosyltransferase activity or activities of LH3 to modify the non-collagenous protein to HMW or other functional form.

Within the scope of the present invention are also treatments where the condition of a person can be improved by adjusting the HMW oligomeric form or other functional form of non-collagenous protein or hydroxylase or glycosyltransferase activity or both activities of LH3 in the blood circulation and/or tissue of the person to a more appropriate level (i.e. a level comparable to the level in healthy person's blood circulation).

In addition to hydroxylase and/or glycosyltransferase activity or activities of LH3 also other lysyl hydroxylases having lysyl hydroxylase and glycosyltransferase (GT and GGT) activities or a fragment or modified form of these having at least one of these activities can be used in the treatments.

According to one preferred embodiment of the invention the HMW oligomeric form or other functional form of specific non-collagenous protein is increased in blood circulation and/or tissue of a patient by the aid of LH3 or other lysyl hydroxylase having lysyl hydroxylase or glycosyltransferase activity or both activities.

According to one preferred embodiment of the invention this can be achieved by posttranslationally modifying and/or oligomerizing the non-collagenous protein outside the human body by using LH3 or other lysyl hydroxylase having lysyl hydroxylase or glycosyltransferase activity or both activities, and administrating HMW form or other functional form of the non-collagenous protein to the blood circulation and/or to the tissue of a patient. The non-collagenous protein is in that case preferably produced as a recombinant protein in a suitable expression system.

According one further preferred embodiment of the invention a nucleic acid sequence encoding LH3 or other lysyl hydroxylase having lysyl hydroxylase or glycosyltransferase activity or both activities is introduced to and expressed in a tissue or cell culture producing a specific non-collagenous protein, thereby increasing the level of lysine modifications and/or the oligomerization of the synthesized non-collagenous protein. The oligomerized form of the non-collagenous protein can be isolated and purified from the tissue culture and administered to the tissue and/or blood circulation of the patient.

According to another preferred embodiment of the invention LH3 or other lysyl hydroxylase having lysyl hydroxylase or glycosyltransferase activity or both activities, is increased in blood circulation and/or tissue of a patient by administrating LH3 or other lysyl hydroxylase having lysyl hydroxylase or glycosyltransferase activity or both activities to the blood circulation and/or to the tissue of a patient.

According to one further preferred embodiment of the invention a nucleic acid sequence encoding LH3 or other lysyl hydroxylase having lysyl hydroxylase or glycosyltransferase activity or both activities, is introduced to and expressed in the cells and/or tissue producing specific non-collagenous protein in the human body, thereby increasing the level of lysine modifications and/or expression, oligomerization and/or secretion of the synthesized non-collagenous protein. For example, in regard to adiponectin, a nucleic acid sequence encoding LH3 or other lysyl hydroxylase having lysyl hydroxylase or glycosyltransferase activity or both activities, is introduced to and expressed in adipose tissue of a patient. Adiponectin is oligomerized in the cells or tissues and secreted into the blood circulation from adipocytes in oligomerized form, thereby increasing the HMW form of adiponectin in the tissue and blood circulation of the patient.

According to yet another preferred embodiment of the invention agents increasing the activity or amount of LH3 or other lysyl hydroxylase having lysyl hydroxylase or glycosyltransferase activity or both activities, are used to increase the level of lysine modifications and/or to oligomerize specific non-collagenous protein. For example in insulin resistant states LH3 or other lysyl hydroxylase having lysyl hydroxylase or glycosyltransferase activity or both activities can be used to modify and oligomerize adiponectin by administering the agents by various routes or by injecting them directly to the adipose tissue.

According one preferred embodiment of the invention, the invention provides a product, which comprises an effective amount of lysyl hydroxylase 3 or other lysyl hydroxylase having lysyl hydroxylase and/or glycosyltransferase activity or activities.

According another preferred embodiment of the invention, the invention provides a product, which comprises a nucleic acid sequence encoding lysyl hydroxylase 3 or other lysyl hydroxylase having lysyl hydroxylase and/or glycosyltransferase activity or activities.

According to one further preferred embodiment of the invention, the invention provides a product, which comprises non-collagenous protein preferably in HMW or other functional form or a nucleic acid sequence encoding the non-collagenous protein.

According to yet another preferred embodiment of the invention, the invention provides a product, which comprises an effective amount of lysyl hydroxylase 3 or other lysyl hydroxylase having lysyl hydroxylase and/or glycosyltransferase activity or activities or a nucleic acid sequence encoding lysyl hydroxylase 3 or other lysyl hydroxylase having lysyl hydroxylase and/or glycosyltransferase activity or activities; and non-collagenous protein preferably in HMW or other functional form or a nucleic acid sequence encoding the non-collagenous protein.

A product comprising lysyl hydroxylase 3 or other lysyl hydroxylase having lysyl hydroxylase and/or glycosyltransferase activity or activities or a nucleic acid sequence encoding lysyl hydroxylase 3 or other lysyl hydroxylase having lysyl hydroxylase and/or glycosyltransferase activity or activities may be administered together with non-collagenous protein preferably in HMW or other functional form or a nucleic acid sequence encoding the non-collagenous protein.

A product comprising non-collagenous protein preferably in HMW or other functional form or a nucleic acid sequence encoding the non-collagenous protein may be administered together with lysyl hydroxylase 3 or other lysyl hydroxylase having lysyl hydroxylase and/or glycosyltransferase activity or activities or a nucleic acid sequence encoding lysyl hydroxylase 3 or other lysyl hydroxylase having lysyl hydroxylase and/or glycosyltransferase activity or activities.

The present invention provides also methods for preparing the product and various uses of the product.

The present invention provides also various methods for diagnosing and treating disorders or conditions related to non-collagenous proteins, in particular related to low amount, abnormal oligomerization and/or dysfunction or non-appropriate levels or function of non-collagenous proteins.

The use of LH3 or other lysyl hydroxylase having lysyl hydroxylase or glycosyltransferase activity or both activities to increase the amount of non-collagenous proteins and/or their HMW (or other functional form) level is a physiological way to treat conditions with reduced non-collagenous protein or their HMW (or other functional form) level.

The present invention can be used to treat any disorders or conditions, which are associated with abnormal amount, abnormal oligomerization and/or dysfunction of non-collagenous proteins.

In particular, the present invention can be used in disorders or conditions associated with abnormal amount, abnormal oligomerization and/or dysfunction of non-collagenous protein, in particular adiponectin, such as insulin resistance, type 2 diabetes mellitus, dyslipidemia, obesity, weight gain, metabolic syndrome, hypertension, cardiovascular diseases, artherosclerosis, coronary heart disease, ischemic heart disease, heart condition, myocardial infarction, cardiac failure, inflammation and inflammatory diseases or is a combination of these disorders or conditions. The present invention can be used in all disorders or conditions, in which the use of functional form of a non-collagenous protein, in particular adiponectin is therapeutically effective.

Next the invention will be examined more closely with the aid of the following detailed description in which reference is made to the appended Figures.

### BRIEF DESCRIPTION OF THE FIGURES

FIG. 1. Domain representation of human LH3 (Salo et al., 2008). Putative N-terminal ER signal sequence (ER, amino acids 1-24) is followed by glycosyltransferase (GT/GGT) domain (25-280), which is responsible for GT/GGT activities and has structural similarities with other glycosyltransferases. Lysyl hydroxylase/prolyl 4-hydroxylase (PKHD) domain (565-738) shares homology with 2-oxoglutarate-dependent dioxygenases and includes a 2-oxoglutarate and ironII-dependent oxygenase domain. Domain borders are approximated by sequence comparisons Amino acids important for activity are indicated in corresponding domain. DXD motif is a typical domain of glycosyltransferases and is also crucial for LH3 activity.

FIG. 2. Total adiponectin levels and oligomeric complex distribution. Total adiponectin levels were measured with ELISA (A, D) and different oligomeric forms of adiponectin were separated by gel filtration chromatography and measured with ELISA from the serum of female (A, B, and C) and male (D, E, F, and G) LH mutant mice. Analyses were done from the serum of the female LH mutant mice at ages B) 9 (n=2 wt, 3 mut) and C) 10 months (n=9 wt, 7 mut) and male mice at ages B) 3.5 months (n=2), C) 8 months (n=2) and D) 10 months (n=7 wt, 5 mut).

FIG. 3. Total adiponectin levels (A) and oligomeric complex distribution in adipose tissue of (B) female (n=5) and (C) male (n=4 wt, 5 mut) LH mutant mice.

FIG. 4. Adiponectin expression in the mRNA (A) and protein (B) level in epididymal adipose tissue of LH mutant female and male mice. The mRNA level was determined with quantitative real time PCR using adiponectin specific TaqMan gene expression assay (Applied Biosystems) and adiponectin protein level was analyzed by Western blot with adiponectin specific antibody and quantified with Quantity One program. The wild type level was set as 100% in figure B. Adipose tissue from heterozygous LH3 knockout mice (LH3 KO+/−) is given for comparison.

FIG. 5. Immunoblot analysis of adiponectin in serum samples of LH mutant and wild type (wt) mice at the age of 5 months (n=7). (A) Equal volumes of LH mutant and wild type serum were loaded into 15% SDS-PAGE gel and adiponectin was detected using adiponectin specific antibody and bands were quantified using ImageQuant TL program. (B) The determination of molecular weight difference between wt and LH mutant adiponectin with immunoblot analysis. Into 15% SDS-PAGE gel 5 μl and 10 μl of 1/300 diluted wt and LH mutant serum samples, respectively, were loaded.

FIG. 6 Immunoblot analysis of adiponectin in adipose tissue homogenate of LH mutant and wt mice (age 6 months, n=6). Equal amount of LH mutant and wt adipose tissue homogenate (41 μg of protein) were loaded into 15% SDS-PAGE gel and adiponectin was detected using adiponectin specific antibody and bands were quantified using ImageQuant TL program.

FIG. 7. Immunoblot analysis of recombinant adiponectin produced in LH mutant skin fibroblasts. Recombinant adiponectin was immunoprecipitated from the cell culture medium of transfected cells and loaded into 15% SDS-PAGE gel and detected with adiponectin specific antibody.

FIG. 8. Distribution of recombinant adiponectin oligomers produced in LH3 knockout MEFs. Different oligomeric forms of adiponectin were separated by gel filtration chromatography and quantified from immunoblots.

### DETAILED DESCRIPTION OF THE INVENTION

The present invention provides products and methods for the treatment of any disorder or condition, which is associated with abnormal amount of non-collagenous protein, or abnormal oligomerization or dysfunction of non-collagenous protein in the blood circulation and/or tissue of a patient.

Present invention comprises that non-collagenous protein and/or HMW or other functional form of the non-collagenous protein is adjusted in the blood circulation and/or tissue of the patient substantially to the level it is in the blood circulation and/or tissue of a healthy person, by using lysyl hydroxylase and/or glycosyltransferase activity or activities to modify the non-collagenous protein to HMW or other functional form.

Present invention comprises also that non-collagenous protein and/or HMW or other functional form of the non-collagenous protein is adjusted in the blood circulation and/or tissue of the patient to more appropriate level, i.e. to a level, which improves the condition of the patient.

By “abnormal amount”, “abnormal oligomerization” or “dysfunction” of non-collagenous protein is meant an amount, oligomerization or function deviating from the amount, oligomerization or function of a healthy person.

By “functional form of the non-collagenous protein” is here meant functional non-collagenous protein, preferably in HMW or other functional form.

By “modifying” is here meant hydroxylation of lysine and/or glycosylation of hydroxylysine residues in non-collagenous protein leading to oligomerized, functional form of non-collagenous protein.

By “a pharmaceutically or therapeutically effective amount of lysyl hydroxylase 3 (LH3) or other lysyl hydroxylase (LH) having lysyl hydroxylase and glycosyltransferase (GT and GGT) activities or a fragment or modified form of these having at least one of these activities is meant an amount capable of modifying non-collagenous protein to HMW or other functional form.

The treatment according to the present invention can be combined with existing therapy for the disorders or conditions. Such therapies include for example anti-obesity or anti-diabetes drugs.

### Non-Collagenous Proteins

The present invention can be applied generally to non-collagenous proteins, in particular to the non-collagenous proteins described below.

There are nearly 20 proteins that are not members of the collagen family, but all these non-collagenous proteins have a short collagenous domain in their structure. By a non-collagenous protein is meant a protein having a collagenous domain with at least 6 Xaa-Yaa-Gly repeats, wherein Xaa and Yaa are any amino acids. Preferably, Yaa is selected from the group comprising 4-hydroxyproline, hydroxylysine, galactosyl hydroxylysine and glycosylgalactosyl hydroxylysine. Preferably, Xaa is proline in the Xaa-Yaa-Gly triplet.

Noncollagenous proteins may comprise up to 160 repeats, some groups of non-collagenous proteins comprise 10 to 100, 10 to 80, or 10 to 60, typically non-collagenous proteins comprise 10 to 40, Xaa-Yaa-Gly repeats.

Noncollagenous proteins can be divided into the following groups of proteins:
1) C1q domain containing proteins with 14 to 153 repeats

Examples are adiponectin, adiponectin like proteins 1 and 2, C1q subcomponent of complement activation, C1qTNF proteins 1-3 (3=CORS26=cartonectin), emilin 1 and 2, Cgliacolin 1 and 2, CRF 1 and 2;
2) Collectins with 17-59 repeats and ficolins with 11-19 repeats

Examples are mannan binding lectin, surfactant protein A and D, collectins L1, P1 and K1. H-ficolin, L-ficolin and M-ficolin.
3) Collagenous transmembrane protein

Examples are class A macrophage scavenger receptor, MARCO, receptor, ectodysplasin, collomin.
4) Acetylcholinesterase, butyrylcolinesterase
5) Collagen triple helix containing protein 1.

Non-collagenous proteins, such as MARCO protein has 89 repeats, macrophage scavenger receptor types I and II 24 repeats, and collagenous tail (collagen Q) of asetylcholinesterase and butyrylcholinesterase 63 repeats.

At least adiponectin, mannan-binding lectin, C1q subcomponent of complement activation and surfactant protein D are known to have Glc-Gal-Hyl residues in their collagenous domain. It is very likely that also other noncollagenous proteins containing hydroxylysine, such as surfactant protein A, collagenous tail collagen Q of asetylcholinesterase and butyrylcholinesterase, conglutinin, collectin-46, can be further glycosylated. Noncollagenous proteins with lysine residue(s) in the Yaa position in the Xaa-Yaa-Gly repeat, such as collectin liver 1 (CL-L1), collectin placenta 1 (CL-P1), collectin kidney 1 (CL-K1), macrophage receptor MARCO, macrophage scavenger receptor types I and II, C1q and tumor necrosis factor related protein (C1qTNF) 1, 2, 3/CORS-26/cartonectin, 5, 6, 7 and 8, otolin-1, adipoQ-like 1 (AQL1), adipoQ-like 2 (AQL2), gliacolin 1 and 2, collagen triple helix repeat containing 1, gliomedin, and CRF 1 and 2, are also very potential candidates to be further hydroxylated and glycosylated since modification of lysine has not been analysed in mentioned non-collagenous proteins.

It is also very probable that the glycosylated hydroxylysines have a functional role in the above mentioned proteins due to the structural and/or functional similarities with adiponectin and/or mannan-binding lectin.

Adiponectin (also known as ACRP30 and AdipoQ), a 30-kDa protein consists from N-terminal variable, a short collagenous domain and C-terminal globular domain. Full-length adiponectin circulates in the plasma as different oligomeric forms: as a trimer (low molecular weight, LMW), a hexamer (middle molecular weight, MMW) and a larger oligomeric structure of high molecular weight (HMW) (Wang et al. 2008). The formation of different oligomeric forms depends on the hydroxylation and glycosylation of four lysine residues in the collagen-like domain and they are essential in the formation of HMW oligomer (Richards et al. 2006; Wang et al. 2006).

Adiponectin is the major insulin-sensitizing hormone secreted by adipose tissue with important anti-diabetic, anti-inflammatory and anti-atherosclerotic functions. Decreased plasma concentrations of adiponectin have a causal role in the development of insulin resistance, type 2 diabetes and metabolic syndrome (Tilg and Moschen 2006). In obesity adipokines are increased, but adiponectin is down-regulated by unknown mechanism. Interestingly, the ratio of HWM to total adiponectin, not the total amount of adiponectin, seems to be clinically more significant determinant in respect of diabetes and coronary artery disease (Szmitko et al. 2007). This is in good agreement with the finding that HMW complex is the most active form of adiponectin in suppressing serum glucose levels via hepatic glucose production (Pajvani et al. 2004). Moreover, HMW adiponectin can protect endothelial cells from apoptosis, whereas trimeric and hexameric forms have no effect (Kobayashi et al. 2004).

In the present invention it is shown (exemplified in LH mutant mice) that the LH3 or LH activity affects directly the modifications of specific lysine residues of adiponectin. The reduction in the molecular weight of adiponectin corresponds with the loss of 1 to 3 Glc-Gal-Hyl residues in the presence of malfunctional LH3.

According to the present invention LH3 or LH activity of LH3 has also an effect on the total adiponectin. Malfunctional LH3 decreases significantly the level of secreted total adiponectin. In adipose tissue adiponectin protein level was about 70 to 90% of the control whereas in serum was significantly decreased when compared with the wild type. This indicates that the secretion of the non-collagenous protein is not normal, since adiponectin accumulates in the adipose tissue.

According to the present invention the LH3 or LH activity of LH3 regulates the formation of different oligomeric forms. Malfunctional LH3 caused significant reduction of HMW form and accordingly significant increase of LMW adiponectin both in adipose tissue and serum.

The more evident reduction of total adiponectin level in serum than in adipose tissue and reduction of HMW adiponectin both in serum and adipose tissue due to malfunctional LH3 indicate that formation of oligomers or seqretion of oligomers is abnormal due to changes in lysine modifications catalyzed by LH3 and thus in the oligomerization.

Within the scope of the present invention are adiponectins from various different origins. Recombinantly produced adiponectin can be oligomerized outside the body of the patient and administrated to the patient in need for the treatment. Adiponectin can be from any origin, if the adiponectin functions in a similar manner as adiponectin from human origin.

Alternatively the production and/or oligomerization of adiponectin can be increased in the body of the patient.

Within the scope of the present invention are adiponectins comprising an amino acid sequence selected from the group consisting of SEQ ID NO: 1 to SEQ ID NO:26, or an amino acid sequence selected from the group consisting of a sequence having at least 80%, preferably at least 85% identity, more preferably at least 90% identity, still more preferably at least 95%, most preferably at least 98% identity to the mature sequence of SEQ ID NO: 1 to SEQ ID NO:26, or a fragment of these sequences having essentially the same function as adiponectin, and nucleic acid sequences encoding said amino acid sequences (see Table 1).

TABLE 1

| Proteins | Protein accession number |
|---|---|
| adiponectin (Sus scrofa) | NP_999535 (SEQ ID NO: 1) |
| adiponectin precursor (Sus scrofa) | CAH61062 SEQ ID NO: 2 |
| adiponectin (Sus scrofa domestica) | AAQ24844 (SEQ ID NO: 3) |
| (domestic pig) | |
| Adiponectin precursor ((Sus scrofa) | AAQ22996 (SEQ ID NO: 4) |
| Oryctolagus cuniculus (rabbit) | NP_001075691 (SEQ ID NO: 5) |
| adiponectin (Homo sapiens) | ABZ10942 (SEQ ID NO: 6) |
| Adiponectin (Anser anser) (domestic goose) | ACA63478(SEQ ID NO: 7) |
| Adiponectin (canis lupus familiaris)((dog) | NP_001006645 XP_535838 (SEQ ID NO: 8) |
| Adiponectin (Macaca fuscata) (Japanese macaque) | BAG16752(SEQ ID NO: 9) |
| Adiponectin (Macaca mulatta) (rhesus monkey) | NP_001028043 (SEQ ID NO: 10) |
| Adiponectin (Gallus gallus) (chicken) | AAX40986 (SEQ ID NO: 11) |
| Adiponectin (Felis catus; domestic cat) | BAF52934 (SEQ ID NO: 12) |
| Adiponectin (Felis catus; domestic cat) | ABG01984 (SEQ ID NO: 13) |
| Adiponectin (Felis catus) | CAG25969 (SEQ ID NO: 14) |
| Adiponectin (Anas platyrhynchos) | ABE03631 (SEQ ID NO: 15) |
| Adiponectin (Vulpes vulpes) | ABD98113 (SEQ ID NO: 16) |
| Adiponectin (Alopex lagopus; Arctic fox) | AAX73247 (SEQ ID NO: 17) |
| Adiponectin (Nyctereutes procyonoides) | AAX73246 (SEQ ID NO: 18) |
| (raccoon dog) | |
| Adiponectin, Q10 and collagen domain | NP_653345 (SEQ ID NO: 19) |
| containing (Rattus norvegicus) | |
| Adiponectin Q10 and collagen domain | NP_033735 (SEQ ID NO: 20) |
| containing (Mus musculus) | |
| Adiponectin precursor (adipocyte, C1q and | Q3Y5Z3 SEQ ID NO: 21) |
| collagen domain-containing protein) | |
| (30 kDa adipocyte complement-related | |
| protein) (adipocyte complement-related 30 | |
| kDa protein) (ACRP30) (adipose most | |
| abundant gene transcript 1 protein) apM-1) | |
| Bos Taurus) (cattle) | |
| Adiponectin, C1Q and collagen domain | NP_001038890 (SEQ ID NO: 22) |
| containing, like (Danio rerio) (zebrafish) | |
| Adiponectin a (Danio rerio) | ABY78995 (SEQ ID NO: 23) |
| Adiponectin b (Danio rerio) | ABY78996 (SEQ ID NO: 24) |
| Adiponectin, C1Q and collagen domain | XP_691074 (SEQ ID NO: 25) |
| containing, like 2(Danio rerio) | |
| Similar to adiponectin | XP_001506649 (SEQ ID NO: 26) |
| (Ornithorhynchus anatinus) (platypus) | |

Amino acid sequences of adiponectin are known from various origins as can be seen in Table 1. In addition the following adiponectin amino acid sequences are known:
DEFINITION adiponectin [Sus scrofa (pig)]
ACCESSION NP_{—} 999535, ACCESSION AAS75592, ACCESSION NP_{—}999535, ACCESSION ABQ95350,
ACCESSION ACB06569, ACCESSION ACB06568, ACCESSION ACB06567,
ACCESSION ACA50588, ACCESSION ABZ85675, ACCESSION ABO30578, ACCESSION AAZ86518,
ACCESSION AAZ86517, ACCESSION AAZ86516, ACCESSION AAQ83880,
ACCESSION AAN11297, ACCESSION AAT00459, ACCESSION AAU87581
DEFINITION adiponectin precursor [Sus scrofa].
ACCESSION CAH61062
DEFINITION adiponectin [Sus scrofa domestica (domestic pig)].
ACCESSION AAQ24844
DEFINITION adiponectin precursor [Sus scrofa].
ACCESSION AAQ22996
DEFINITION adiponectin [Oryctolagus cuniculus (rabbit].
ACCESSION NP_{—}001075691
DEFINITION adiponectin [Oryctolagus cuniculus].
ACCESSION ABC60052
DEFINITION adiponectin [Homo sapiens].
ACCESSION ABZ10942
DEFINITION adiponectin precursor [Homo sapiens].
ACCESSION NP_{—}004788
DEFINITION unnamed protein product [Homo sapiens].
ACCESSION BAF84214
DEFINITION Adiponectin, C1Q and collagen domain containing [Homo sapiens].
ACCESSION AAH96311, ACCESSION AAH96310, ACCESSION AAH96308, ACCESSION AAH96309,
ACCESSION EAW78165
DEFINITION Adiponectin precursor (Adipocyte, C1q and collagen domain-containing protein) (30 kDa adipocyte complement-related protein) (Adipocyte complement-related 30 kDa protein) (ACRP30) (Adipose most abundant gene transcript 1 protein) (apM-1) (Gelatin-binding protein).
ACCESSION Q15848
DEFINITION ADIPOQ protein [Homo sapiens].
ACCESSION AAH54496
DEFINITION adiponectin [Anser anser (domestic goose)].
ACCESSION ACA63478
DEFINITION adiponectin [Canis lupus familiaris (dog)].
ACCESSION NP_{—}001006645 XP_{—}535838
DEFINITION adiponectin [Canis familiaris].
ACCESSION BAD15362, ACCESSION AAL09702
DEFINITION adiponectin [Macaca fuscata (Japanese macaque)].
ACCESSION BAG16752
DEFINITION adiponectin [Macaca mulatta (rhesus monkey)].
ACCESSION NP_{—}001028043
DEFINITION adiponectin [Macaca mulatta].
ACCESSION AAK92202
DEFINITION adiponectin [Gallus gallus (chicken)].
ACCESSION AAX40986
DEFINITION adiponectin [Gallus gallus].
ACCESSION AAV48534
DEFINITION adiponectin [Gallus gallus].
ACCESSION AAS67924
DEFINITION adiponectin, C1Q and collagen domain containing [Gallus gallus].
ACCESSION NP_{—}996874
DEFINITION adiponectin [Felis catus (domestic cat)].
ACCESSION BAF52934
DEFINITION adiponectin, C1Q and collagen domain containing [Felis catus].
ACCESSION NP_{—}001078907
DEFINITION adiponectin [Felis catus (domestic cat)].
ACCESSION ABG01984
DEFINITION adiponectin [Felis catus].
ACCESSION CAG25969
DEFINITION adiponectin [Anas platyrhynchos].
ACCESSION ABE03631
DEFINITION adiponectin [Vulpes vulpes (red fox)].
ACCESSION ABD98113
DEFINITION adiponectin [Alopex lagopus (Arctic fox)].
ACCESSION AAX73247
DEFINITION adiponectin [Nyctereutes procyonoides (raccoon dog)].
ACCESSION AAX73246
DEFINITION adiponectin, C1Q and collagen domain containing [Rattus norvegicus].
ACCESSION NP_{—}653345
DEFINITION Adiponectin, C1Q and collagen domain containing [Rattus norvegicus].
ACCESSION AAH92565
DEFINITION 30 kDa adipocyte complement-related protein [Rattus norvegicus].
ACCESSION AAK61608
DEFINITION adiponectin, C1q and collagen domain containing [Rattus norvegicus].
ACCESSION EDL78080
DEFINITION adiponectin, C1Q and collagen domain containing [Mus musculus].
ACCESSION NP_{—}033735
DEFINITION Adiponectin precursor (Adipocyte, C1q and collagen domain-containing protein) (30 kDa adipocyte complement-related protein) (Adipocyte complement-related 30 kDa protein) (ACRP30) (Adipocyte-specific protein AdipoQ). Mus musculus (house mouse)
ACCESSION Q60994
DEFINITION adiponectin, C1Q and collagen domain containing [Mus musculus].
ACCESSION EDK97661
DEFINITION Adiponectin, C1Q and collagen domain containing [Mus musculus].
ACCESSION AAH28770
DEFINITION 30 KDa adipocyte complement-related protein [Mus musculus].
ACCESSION AAW82905
DEFINITION 30 kDa adipocyte complement-related protein [Mus musculus].
ACCESSION AAW70555
DEFINITION adipocyte complement-related protein [Mus musculus].
ACCESSION AAK13417
DEFINITION adipoQ [Mus musculus].
ACCESSION AAB06706
DEFINITION Adiponectin precursor (Adipocyte, C1q and collagen domain-containing protein) (30 kDa adipocyte complement-related protein) (Adipocyte complement-related 30 kDa protein) (ACRP30) (Adipose most abundant gene transcript 1 protein) (apM-1). Bos taurus (cattle)
ACCESSION Q3Y5Z3
DEFINITION ADIPOQ protein [Bos taurus].
ACCESSION AAI40489
DEFINITION adiponectin, C1Q and collagen domain containing, like [Danio rerio (zebrafish)].
ACCESSION NP_{—}001038890
DEFINITION Adiponectin, C1Q and collagen domain containing, like [Danio rerio].
ACCESSION AAI22339
DEFINITION adiponectin b [Danio rerio].
ACCESSION ABY78996
DEFINITION adiponectin a [Danio rerio].
ACCESSION ABY78995
DEFINITION PREDICTED: adiponectin, C1Q and collagen domain containing, like 2 [Danio rerio].
ACCESSION XP_{—}691074
DEFINITION PREDICTED: similar to adiponectin [Ornithorhynchus anatinus (platypus)].
ACCESSION XP_{—}001506649

### Lysyl Hydroxylase 3 or Other Lysyl Hydroxylase Having Lysyl Hydroxylase or Glycosyltransferase Activity or Both Activities

Lysyl hydroxylase 3 (LH3) is a multifunctional, post-translational enzyme, which possesses lysyl hydroxylase (LH EC 1.14.11.4), glucosyltransferase (GGT, EC 2.4.1.66) and galactosyltransferase (GT, EC 2.4.1.50) activities. Galactosyltransferase (GT) and glucosyltransferase (GGT) activities are called also glycosyltransferase activities. The active sites of GT/GGT and LH activity are distributed into amino- and carboxy terminal ends of the LH3 molecule, respectively (Myllyla et al. 2007). LH3 is located in cells in the endoplasmic reticulum, but in addition to that it is found also in extracellular space and in serum.

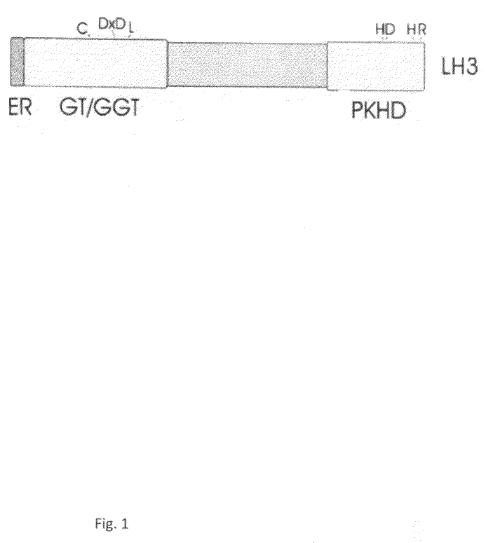

Salo et al., 2008 have described domain representation of human LH3 (see FIG. 1). Putative N-terminal ER signal sequence (ER, amino acids 1-24) is followed by glycosyltransferase (GT/GGT) domain (25-280), which is responsible for GT/GGT activities and has structural similarities with other glycosyltransferases. Lysyl hydroxylase/prolyl 4-hydroxylase (PKHD) domain (565-738) shares homology with 2-oxoglutarate-dependent dioxygenases and includes a 2-oxoglutarate and ironII-dependent oxygenase domain. Domain borders are approximated by sequence comparisons. Amino acids important for activity are indicated in corresponding domain. DXD motif is a typical domain of glycosyltransferases and is also crucial for LH3 activity. Lysyl hydroxylases having lysyl hydroxylase and glycosyltransfease activities from other origin have corresponding domain structure.

In some species lysyl hydroxylase activity is encoded by three genes. In these cases, isoform 3, LH3, has glycosyltransferase activity, but the other isoforms LH1 and LH2, do not have this activity. In species, where lysyl hydroxylase activity is encoded by one gene, there is only one form of lysyl hydroxylase, LH, and it has also glycosyltransferase activity.

Within the scope of the present invention are lysylhydroxylase 3 (LH3) and other lysyl hydroxylases (LH), which in their natural form have both lysyl hydroxylase and glycosyltransferase activities. The enzyme may be used in its natural form having both lysyl hydroxylase and glycosyltransferase activities, or a fragment of the enzyme having lysyl hydroxylase or glycosyltransferase activity or both activities, or as modified to have either lysyl hydroxylase or glycosyltransferase activities. More specifically, the enzyme lysyl hydroxylase 3 (LH3) or other lysyl hydroxylase (LH) having lysyl hydroxylase and glycosyltransferase (GT and GGT) activities or a fragment or modified form of these having all or one or two of these activities may be used in the invention.

“Lysyl hydroxylase 3 (LH3) or other lysyl hydroxylase (LH) having lysyl hydroxylase and/or glycosyltransferase activity or activities” means here lysyl hydroxylase 3 (LH3) or other lysyl hydroxylase (LH) having lysyl hydroxylase and glycosyltransferase (GT and GGT) activities or a fragment or modified form of these having all or one or two of these activities.

Within the scope of the present invention are thus LH3 or LH enzyme having in its natural form both lysyl hydroxylase and glycosyltransferase activities, said enzyme comprising an amino acid sequence selected from the group consisting of SEQ ID NO: 27 to SEQ ID NO: 52, or an amino acid sequence selected from the group comprising a sequence having at least 80%, preferably at least 85% identity, more preferably at least 90% identity, still more preferably at least 95%, most preferably at least 98% identity to the mature sequence of SEQ ID NO: 20 to SEQ ID NO:42, or a fragment or modified form of these sequences having lysyl hydroxylase and/or glycosyltransferase activity or activities.

The enzyme or a fragment of the enzyme may lack signal sequence.

By “a nucleic acid sequence encoding lysyl hydroxylase enzyme or a fragment of lysyl hydroxylase enzyme having lysyl hydroxylase and/or glycosyltransferase activities” is meant here a nucleic acid sequence encoding lysyl hydroxylase enzyme isoform 3 (LH3), or a fragment thereof having lysyl hydroxylase or glycosyltransferase or both activities, or encoding lysyl hydroxylase (LH) or a fragment or modified form thereof having lysyl hydroxylase or glycosyltransferase or both activities (in species where only one isoform of LH is available).

By “modified form of the enzyme” or “modified form of the amino acid sequence” is here meant in particular that the nucleic acid sequence encoding the enzyme is genetically modified, but that the enzyme has lysyl hydroxylase or glycosyltransferase or both activities.

The inventors of the present invention have shown that type IV and VI collagen indicate that the LH3 is the molecule affecting the secretion of these highly glycosylated collagen types. They have indicated that intracellular tetramerization of type VI collagen is dependent on LH3 activities. Before the present invention LH3 was not known to also modify non-collagenous proteins. In order to test whether LH3 modifies non-collagenous proteins adiponectin level was measured from the serum of LH mutant mice. In vitro mutagenesis data have indicated that hydroxylation and glycosylation of the four conserved lysines in the collagenous domain of adiponectin have a significant role in the formation of oligomeric forms of adiponectin (Richards et al. 2006; Wang et al. 2006). Disruption of the collagenous domain selectively abrogated the intracellular assembly of the HMW oligomers, which have the highest carbohydrate content (Wang et al. 2006).

In the present invention it has been shown for the first time that lysyl hydroxylase enzyme having lysyl hydroxylase or glycosyltransferase or both activities or fragments or modified forms of these are capable of modifying lysyl residues in non-collagenous proteins.

According to one preferred embodiment of the invention a specific non-collagenous protein is produced in the presence of LH3, or other lysyl hydroxylase having lysyl hydroxylase and/or glycosyltransferase activities, to synthesize an oligomerized form, preferably HMW, or other functional form of the non-collagenous protein in a cellular system, i.e. in a cell or tissue culture.

Recombinant non-collagenous protein can be produced as e.g. FLAG fusion protein, in a suitable expression system, such as a mammalian expression system. LH3 or LH or fragments or modified forms thereof can be added into the medium as recombinant or synthesized proteins. Alternatively the cells can be co-transfected or stably transfected to produce LH3 or LH or fragments or modified forms thereof in the expression system.

Recombinant LH3 or LH can be produced in a form having all three enzyme activities (LH, GT/GGT), or LH3 or LH fragment or other modified form having the activity required for non-collagenous protein oligomerization.

Insect cells can be used as expression system, for example Baculo virus expression system, or eukaryotic cells with for example mammalian expression systems.

Recombinantly produced enzyme can be purified with methods well known for a person skilled in the art, for example with nickel affinity column when produced as his-tagged enzyme.

In the present invention, LH3 or other LH or fragments or modified forms thereof having lysyl hydroxylase and/or glycosyltransferase activity or activities, can be used preferably as synthetically produced or by recombinant methods.

According to another preferred embodiment of the invention purified recombinant LH3 or LH or fragment thereof can be administrated directly into a patient by various routes, such as oral, intravenous, intramuscular, subcutaneous or direct tissue injection, or by using gene therapeutic methods.

Agents increasing the activity or amount of LH3 (or other LH) can be used to enhance oligomerization of non-collagenous proteins, such as adiponectin. These agents can be administrated orally, intravenously, intramuscularly, subcutaneously or by direct injection to the tissue producing the non-collagenous protein. In case of adiponectin the agents can be for example injected to the adipose tissue.

LH3 or other LH or the nucleic acid sequence encoding LH3 or other LH may originate from any source, for example from eukaryote, from mammalian, from insect origin or even from nematodes and metazoa. The enzyme may originate for example from human, bovine, porcine, monkey, dog, horse, chicken, rat, mouse, nematode, zebrafish, fly or platypus origin. Suitable sources are organisms having collagen or protein having collagenous domain or collagen-type protein, or it may be an organism not having the mentioned collagen proteins, but still producing lysyl hydroxylase enzyme, which has lysyl hydroxylase and glycosyltransferase activities. The nucleotide sequence may be synthetic or at least partly synthetic. Within the scope of the invention are also nucleotide sequences encoding lysyl hydroxylases isolated from new organism groups provided that the nucleotide sequence encodes also an enzyme having lysyl hydroxylase and/or glycosyltransferase activities. The nucleotide and amino acid sequences and fragments and mutants thereof of human LH3, mouse LH3 and C. elegans are described for example in WO 01/92505.

Within the scope of the present invention are thus LH3 or other LH enzyme having in its natural form both lysyl hydroxylase and glycosyltransferase activities, said enzyme comprising an amino acid sequence selected from the group consisting of SEQ ID NO: 27 to SEQ ID NO: 52, or an amino acid sequence selected from the group consisting of a sequence having at least 80%, preferably at least 85% identity, more preferably at least 90% identity, still more preferably at least 95%, most preferably at least 98% identity to the mature sequence of SEQ ID NO: 27 to SEQ ID NO:52, or a fragment or modified form of these sequences having both lysyl hydroxylase and/or glycosyltransferase activities. Preferably, the identity is measured by comparing to the mature protein sequence without signal peptide.

The enzyme or fragment thereof may be used with or without signal sequence.

In Table 2 has been presented amino acid and nucleotide sequence accession numbers of currently available amino acid and nucleotide sequences of LH3 and LH enzymes.

In Table 3 has been presented the amino acid and nucleotide sequence accession numbers of amino acid sequences and nucleic acid sequences of proteins similar to LH3 or LH, but which are not called LH3 or LH.

In Table 4 has been presented amino acid and nucleotide sequence accession numbers of amino acid sequences and nucleic acid sequences of proteins which are not called LH3 or LH, but which have DYnD motif or Cysteine in a corresponding position as Cysteine is in human LH3 amino acid sequence. In human LH3 Cysteine is at position 144.

Many glycosyltransferase families have a DYnD motif in their sequence, which is thought to have a role in Mn²⁺ binding and catalysis of glycosylation reaction (Unligil et al. 2000). Mutagenesis analysis has shown that Cys-144 is required for glycosyltransferase activities of LH3 (Wang et al. 2002b, Wang et al. 2002c).

Amino acid sequences of lysyl hydroxylases useful in this invention are known from various origin as can be seen in Tables 2 to 4. In addition the following LH3 or LH amino acid sequences are known:
DEFINITION lysyl hydroxylase 3 [Mus musculus].
ACCESSION AAK00576
DEFINITION lysyl hydroxylase 3 [Mus musculus].
ACCESSION AAD54618 mus musculus
DEFINITION procollagen-lysine, 2-oxoglutarate 5-dioxygenase 3 [Mus musculus]
ACCESSION NP_{—}036092
DEFINITION Procollagen-lysine, 2-oxoglutarate 5-dioxygenase 3 precursor (Lysyl hydroxylase 3) (LH3).
ACCESSION Q9R0E1
DEFINITION lysyl hydroxylase 3 [Takifugu rubripes (Fugu rubripes)].
ACCESSION NP_{—}001093075
DEFINITION lysyl hydroxylase 3 [Takifugu rubripes]
ACCESSION BAF61137
DEFINITION lysyl hydroxylase 3 [Homo sapiens].
ACCESSION AAF63701
DEFINITION lysyl hydroxylase 3 [Homo sapiens].
ACCESSION AAC34808
DEFINITION lysyl hydroxylase 3 [Homo sapiens].
ACCESSION AAD45831
DEFINITION lysyl hydroxylase 3 [Homo sapiens].
ACCESSION AAO61775
DEFINITION procollagen-lysine, 2-oxoglutarate 5-dioxygenase 3 precursor [Homo sapiens].
ACCESSION NP_{—}001075
DEFINITION Procollagen-lysine, 2-oxoglutarate 5-dioxygenase 3 precursor (Lysyl hydroxylase 3) (LH3).
ACCESSION O60568
DEFINITION Procollagen-lysine, 2-oxoglutarate 5-dioxygenase 3 precursor (Lysyl hydroxylase 3) (LH3)_HUMAN
DEFINITION lysyl hydroxylase isoform 3 [Homo sapiens
ACCESSION AAC39753
DEFINITION Procollagen-lysine, 2-oxoglutarate 5-dioxygenase 3 precursor (Lysyl hydroxylase 3) (LH3)_RAT.
ACCESSION Q5U367
DEFINITION procollagen-lysine, 2-oxoglutarate 5-dioxygenase 3 [Rattus norvegicus].
ACCESSION CAD23628
DEFINITION procollagen-lysine, 2-oxoglutarate 5-dioxygenase 3 [Rattus norvegicus].
ACCESSION NP 835202
DEFINITION Procollagen-lysine, 2-oxoglutarate 5-dioxygenase 3 precursor (Lysyl hydroxylase 3) (LH3). Pongo abelii (Sumatran orangutan)
ACCESSION Q5R6K5
DEFINITION PREDICTED: similar to lysyl hydroxylase isoform 3 [Pan troglodytes (chimpanzee)].
ACCESSION XP 001142249
DEFINITION PREDICTED: similar to procollagen-lysine, 2-oxoglutarate 5-dioxygenase 3 precursor isoform 1 [Canis lupus familiaris (dog)].
ACCESSION XP 536856
DEFINITION PREDICTED: similar to procollagen-lysine, 2-oxoglutarate 5-dioxygenase 3 precursor isoform 2 Canis lupus familiaris (dog)
ACCESSION XP_{—}858413
DEFINITION procollagen lysine 2-oxoglutarate 5-dioxygenase 3 [Danio rerio (zebrafish)].
ACCESSION AAY84150
DEFINITION procollagen-lysine 2-oxoglutarate 5-dioxygenase 3 [Danio rerio (zebrafish)].
ACCESSION NP_{—}001037808 XP_{—}683880
DEFINITION PREDICTED: hypothetical protein isoform 6 [Bos Taurus (cattle)].
ACCESSION XP_{—}887254
DEFINITION procollagen-lysine, 2-oxoglutarate 5-dioxygenase 3 [Xenopus Laevis (African clawed frog)].
ACCESSION NP_{—}001080446
DEFINITION Procollagen-lysine, 2-oxoglutarate 5-dioxygenase precursor (Lysyl hydroxylase) (LH) (Lethal protein 268) Caenorhabditis elegans.
ACCESSION Q20679
DEFINITION CG6199 CG6199-PA, isoform A [Drosophila melanogaster (fruit fly)].
ACCESSION NP 648451
DEFINITION CG6199 CG6199-PB, isoform B [Drosophila melanogaster].
ACCESSION NP_{—}729687
DEFINITION procollagen-lysine, 2-oxoglutarate 5-dioxygenase [Aedes aegypti (Stegomyia aegypti].
ACCESSION XP_{—}001653115
DEFINITION Probable procollagen-lysine, 2-oxoglutarate 5-dioxygenase (Lysyl hydroxylase) (LH). Acanthamoeba polyphaga mimivirus
ACCESSION Q5UQC3
DEFINITION Procollagen-lysine, 2-oxoglutarate 5-dioxygenase precursor, putative [Brugia malayi].
ACCESSION EDP31117
DEFINITION PREDICTED: similar to procollagen-lysine, 2-oxoglutarate 5-dioxygenase 3 [Ornithorhynchus anatinus (platypus)].
ACCESSION XP_{—}001516384
DEFINITION PREDICTED: similar to procollagen-lysine, 2-oxoglutarate 5-dioxygenase [Nasonia vitripennis (jewel wasp)].
ACCESSION XP_{—}001601697
DEFINITION predicted protein [Nematostella vectensis (starlet sea anemone)].
ACCESSION XP_{—}001633491
DEFINITION PREDICTED: hypothetical protein [Equus caballus (horse)].
ACCESSION XP_{—}001504506
DEFINITION AGAP001507-PA [Anopheles gambiae str. PEST].
ACCESSION XP_{—}321614
DEFINITION Hypothetical protein CBG13377 [Caenorhabditis briggsae AF16].
ACCESSION XP_{—}001678516
DEFINITION GA19434-PA [Drosophila pseudoobscura].
ACCESSION XP_{—}001353930

The nucleotide sequence of the full-length human LH3 (coding sequence) is presented in the Sequence Listing as SEQ ID NO:53

TABLE 2

| Proteins | Protein accession number | Nucleotide accession number |
|---|---|---|
| Human LH3 (DDDDD₁₈₇₋₁₉₁ | NP_001075 (SEQ ID NO: 27 | NM_001084 |
| motif and Cys₁₄₄) | | |
| Human LH3 (DDDDD₁₈₇₋₁₉₁ | AAF63701 (SEQ ID NO: 28) | |
| motif and Cys₁₄₄) | | |
| Bos taurus (cattle) | XP_887254 (SEQ ID NO: 29) | XM_882161 |
| hypothetical protein isof. 6 | | |
| (DDDDD₂₀₀₋₂₀₄ motif and | | |
| Cys₁₅₇) | | |
| Dog LH3 (DDDDD₁₈₉₋₁₉₃ | XP_536856 (LH3 precursor isoform | XM_536858 (LH3 precursor |
| motif and Cys₁₄₆) | 1) (SEQ ID NO: 30) | isoform 1) |
| | XP_858413 (LH3 precursor isoform | XM_853320 (LH3 precursor |
| | 2) (SEQ ID NO: 31) | isoform 2) |
| Mus musculus LH3 | AAK00576 (SEQ ID NO: 32) | |
| (DDDDD₁₉₀₋₁₉₄ motif and Cys₁₄₇) | | |
| Mouse LH3 (DDDDD₁₉₀₋₁₉₄ | Q9R0E1 | AF046783 |
| motif and Cys₁₄₇) | | |
| Rat LH3 (DDDDD₁₉₀₋₁₉₄ | CAD23628 (SEQ ID NO: 33) | AJ430859 |
| motif and Cys₁₄₇) | | |
| Rat LH3 (DDDDD₁₉₀₋₁₉₄ | Q5U367 (SEQ ID NO: 34) | |
| motif and Cys₁₄₇) | | |
| Sumatran orangutan | Q5R6K5 (SEQ ID NO: 35) | |
| LH3(DDDDD₁₈₇₋₁₉₁ motif and Cys₁₄₄) | | |
| Danio rerio | AAY84150 (SEQ ID NO: 36) | |
| (zebrafish)(DDDDD₁₇₆₋₁₈₀ | | |
| motif and Cys₁₃₃) | | |
| Zebrafish LH3 (DDDDD₁₇₆₋₁₈₀ | NP_001037808(SEQ ID NO: 37) | NM_001044343 |
| motif and Cys₁₃₃) | | |
| Xenopus laevis (African frog) | NP_001080446 (SEQ ID NO: 38) | NM_001086977 |
| LH3 (DNDDD₁₈₁₋₁₈₅ motif and Cys₁₃₈) | | |
| Takifugu rubripes LH3 | NP_001093075(SEQ ID NO: 39) | NM_001099605 |
| (DNDDD₁₇₉₋₁₈₃ motif and Cys₁₃₆) | | |
| C. elegans (Lethal protein | Q20679 (SEQ ID NO: 40) | Z66512 |
| 268) LH(DKDDD₁₇₅₋₁₇₉ motif and Cys₁₃₂) | | |
| Drosophila melanogaster LH | NP_648451 (isoform A) (SEQ ID NO: 41) | NM_140194 (isoform A) |
| (DTADD₁₇₆₋₁₈₀ motif and Cys₁₃₃) | NP_729687 (isoform B) | NM_168452 (isoform B) |
| Aedes aegypti LH | XP_001653115(SEQ ID NO: 42) | XM_001653065 |
| (DTDDD₁₆₁₋₁₆₅ motif and Cys₁₁₈) | | |
| Acanthamoeba polyphage | Q5UQC3(SEQ ID NO: 43) | YP_142584 |
| mimivirus LH (no DYnD motif and no Cys) | | |
| Brugia malayi LH precursor; putative | EDP31117(SEQ ID NO: 44) | DS239414 |
| (DNDDD₁₈₁₋₁₈₅ motif and Cys₁₃₈) | | |

TABLE 3

| Proteins | Protein accession number | Nucleotide accession number |
|---|---|---|
| Ornithorhynchus anatinus LH3 | XP_001516384(SEQ ID NO: 45) | XM_001516334 |
| (similar to LH3) (DDDDD₁₉₂₋₁₉₆ | | |
| motif and Cys₁₄₇) | | |
| Nasonia vitripennis (jewel asp) | XP_001601697(SEQ ID NO: 46) | XM_001601647 |
| (similar to LH) (DDDDD₁₇₄₋₁₇₈ | | |
| motif and Cys₁₃₁) | | |
| Monodelphis domestica | XP_001371229 | XM_001371192 |
| (similar to LH) (DDDDD₂₁₃₋₂₁₇ | | |
| and Cys₁₇₀) | | |
| Pan troglodytes (chimpanzee) | XP_001142249 (SEQ ID NO: 47) | |
| similar to LH3 (DSDSD₁₆₂₋₁₆₆ | | |
| motif, no Cys) | | |

TABLE 4

| Proteins | Protein accession number | Nucleotide accession number |
|---|---|---|
| Nematostella vectensis (starlet | XP_001633491(SEQ ID NO: 48) | XM_001633441 |
| sea anemone) predicted protein | | |
| (DEDDD₁₈₄₋₁₈₈ motif and Cys₁₄₁) | | |
| Equus caballus (horse) | XP_001504506 (SEQ ID N0: 49) | XM_001504456 |
| hypothetical protein LOC100059479 | | |
| (DDDDD₁₉₂₋₁₉₆ motif and Cys₁₄₉) | | |
| Tetraodon nigroviridis: | CAF89795 | CAAE01007145 |
| unnamed protein product | | |
| (DDDDD₁₇₇₋₁₈₁ motif and Cys₁₀₇) | | |
| Anopheles gambiae str. PEST: | XP_321614 (SEQ ID NO: 50) | XM_321614 |
| AGAP001507-PA (DAEDD₁₆₄₋₁₆₈ | | |
| motif and Cys₁₂₁) | | |
| Tribolium castaneum: similar to | XP_973819 | XM_968726 |
| CG6199-PA, isoform A | | |
| (DTDDD₁₉₈₋₂₀₂ motif and Cys₁₅₅) | | |
| Caenorhabditis briggsae | XP_001678516 (SEQ ID NO: 51) | XM_001678464 |
| AF16; hypothetical protein | | |
| CBG13377 (DKDDD₁₇₇₋₁₈₁ motif | | |
| and Cys₁₃₄) | | |
| Drosophilla psedoobscura: | XP_001353930(SEQ ID NO: 52) | XM_001353894 |
| GA19434-PA (DMDDD₁₅₃₋₁₅₇ | | |
| motif and Cys₁₁₀) | | |
| Strongylocentrotus purpuratus: | XP_001181677 | XM_001181677 |
| similar to Plod-prov protein, | | |
| partial (DIADD₁₁₇₋₁₂₁ motif, no Cys) | | |

The enzyme may be full length LH3 or LH, or a fragment of LH3 or LH, having lysyl hydroxylase and/or glycosyltransferase activities. The enzyme or fragment of enzyme may be with or without signal peptide. For example antibodies are raised against the mature protein without signal peptide.

By the term “identity” is here meant the identity between two amino acid sequences compared to each other from the first amino acid encoded by the corresponding gene to the last amino acid. Preferably the identity is measured by comparing the amino acid sequences without sequences of the signal peptide. The identity of the full-length sequences is measured by using Needleman-Wunsch global alignment program at EMBOSS (European Molecular Biology Open Software Suite; Rice et al., 2000) program package, version 2.9.0, with the following parameters: EMBLOSUM62, Gap penalty 10.0, Extend penalty 0.5.

An “isolated” or “purified” polypeptide, protein or enzyme is substantially free of cellular material or other contaminating proteins from the cell or tissue source from which the protein is derived, or substantially free from chemical precursors or other chemicals when chemically synthesized. In one embodiment, the language “substantially free” means preparation of the protein or enzyme having less than about 30%, less than about 20%, less than about 10% and more preferably less than about 5%, less than about 1% (by dry weight), of contaminating proteins or chemicals or chemical precursors or culture medium, if the protein is recombinantly or synthetically produced.

A “non-essential” amino acid residue is a residue that can be altered from the wild-type sequence or without abolishing or more preferably, without substantially altering a biological activity, whereas an “essential” amino acid residue results in such a change. For example, amino acid residues that are conserved among the polypeptides of the present invention are predicted to be particularly unamenable to alteration.

A “conservative amino acid substitution” is one in which the amino acid residue is replaced with an amino acid residue having a similar side chain. Families of amino acid residues having similar side chains are known in the art. These families include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine, tryptophan), uncharged polar side chains (e.g., glycine, asparagine, glutamine, serine, threonine, tyrosine, cysteine), beta-branched side chains (e.g., threonine, valine, isoleucine) and aromatic side chains (e.g., tyrosine, phenylalanine, tryptophan, histidine). A predicted nonessential amino acid residue is preferably replaced with another amino acid residue from the same side chain family. Alternatively, in another embodiment, mutations can be introduced randomly along all or part of the coding sequence. The resultant mutants can be screened for biological activity to identify mutants that have the desired activity. Following mutagenesis the encoded protein can be expressed recombinantly and the activity of the protein can be determined

Biologically active fragments or portions of the protein or enzyme of the present invention include peptides comprising amino acid sequences sufficiently identical to or derived from the amino acid sequence of the protein.

In particular by a fragment or portion of LH3 or LH enzyme is meant here a fragment or portion of said enzyme having biological or antigenic activity.

By biological activity is meant here in particular that the enzyme or fragment or portion of the enzyme has lysyl hydroxylase and/or glycosyltransferase activities. The enzyme or fragment or portion of the enzyme may have glucosyltransferase or galactosyltransferase activities, or both of these activities.

The fragment of LH3 or LH is any fragment lacking at least one amino acid compared to the full-length polypeptide. In addition the fragment may lack the signal peptide.

The enzymes, biologically active or antigenic fragments of the enzymes, antibodies against the enzymes or against the fragments of the enzymes, Fab fragments of the enzymes, or nucleotide sequences encoding the enzymes or fragments thereof are useful, as reagents or targets in assays applicable to treatment and diagnosis of conditions or disorders, which are associated with abnormal amount of non-collagenous proteins, or abnormal oligomerization or dysfunction of non-collagenous proteins in the blood circulation and/or tissue of a patient. These conditions or disorders can be affected by lysyl hydroxylase and/or glycosyltransferase activities. The presence or absence or level of the lysyl hydroxylase and/or glycosyltransferase activity can be used in assays for diagnosis.

Assays for LH, GT and GGT activity are described in Kivirikko and Myllyla, 1982 (LH activity) and Myllyla et al. 1975 (GT and GGT activity). For a person skilled in the art it is easy to study, whether an enzyme or fragment or portion of enzyme has LH, GT or GGT activity. Also methods for studying these activities from blood or tissue are available.

The term “antibody” as used herein refers to an immunoglobulin molecule or immunologically active portion thereof, i.e., an antigen-binding portion. Examples of immunologically active portions of immunoglobulin molecules include scFV and dcFV fragments, Fab and F(ab′)₂ fragments which can be generated by treating the antibody with an enzyme such as papain or pepsin, respectively.

LH3 or LH enzyme typically comprises a short Aspartic Acid (D) rich conserved motif DYnD responsible for glycosyltransferase activity. In the motif Y typically comprises 1, 2 or 3 amino acids, which vary in different species. For example in human LH3 the motif is DDDDD, whereas in insect Drosophila LH the motif is DTADD. When at least one Aspartic Acid in the D-rich motif is genetically changed not to be Aspartic Acid (D), the enzyme may lose partly or completely its glycosyltransferase activity.

According to one embodiment of the invention the enzyme comprises a conserved motif DYnD motif, wherein D is Aspartic Acid, n is 1, 2 or 3 and Yn means D or any amino acid, the same or different amino acid compared to each other; or

DY₁Y₂Y₃D motif, wherein Y₁ is any amino acid, preferably D, T, K or N. Also I, M, A, E or even S are possible;
Y₂ is any amino acid, preferably D or A. Also E is possible;
Y₃ is any amino acid, preferably D. Also S is possible.

Thus, for example motifs DY₁D, DY₁Y₂D and DY₁Y₂Y₃D are possible, or DY₁DDD or DY₁Y₂DD.

The DYnD motif is responsible for glycosyltransferase activity. If at least one of the Aspartic Acids in the motif is genetically changed not to be Aspartic Acid, the protein or enzyme loses partly or completely its glycosyltransferase activity.

In human LH3 the D-rich motif DDDDD is in the sequence SEQ ID NO:27 (human LH3) at position 187-191, whereas in LH3 or LH from other species D-rich region may be in a corresponding, although slightly different position (see Table 2).

According to one embodiment of the invention the enzyme comprises an amino acid sequence, where Cysteine is in position 144 in SEQ ID NO:27 or in a corresponding position in another LH3 or LH sequence, said Cysteine being responsible for glycosyltransferase activity.

Amino acids responsible for the lysyl hydroxylase activity reside in the carboxy terminus of the protein. The catalytic center of 2-oxoglutarate dioxygenases is composed of His¹-X-Asp/Glu-Xn-His²-motif where X is any amino acid and n is number of amino acids, which varies from 40 to 150. Amino acids His-667 and His-719 in human LH3 correspond to His¹ and His² in the motif. In addition Asp-669 and Agr-729 are required for the lysyl hydroxylase activity.

Useful variations of the above enzymes or fragments of enzymes are enzymes or fragments that are sufficiently or substantially identical to the amino acid sequences shown here.

“Antibodies against LH3 or LH enzyme or against a fragment, such as against the N-terminal part of LH3 or LH enzyme or” “Fab fragments” of the antibodies can be prepared by conventional methods well known to a person skilled in the art.

It may be of advantage, if the enzyme used to treat human is from human origin. However, LH3 or LH from different species can also be used to treat human.

### Administration

The present invention provides a product for the treatment of any disorder or condition, which is associated with abnormal amount of non-collagenous protein, or abnormal oligomerization or dysfunction of non-collagenous protein in the blood circulation and/or tissue of a patient. The treatment comprises a step of non-collagenous protein and/or HMW or other functional form of said non-collagenous protein being adjusted in the blood circulation and/or tissue of the patient substantially to the level it is in the blood circulation and/or tissue of a healthy person or to a more appropriate level for the patient, by using lysyl hydroxylase and/or glycosyltransferase activity or activities to modify the non-collagenous protein to HMW or other functional form.

The present invention provides a product, which preferably comprises:
an effective amount of lysyl hydroxylase 3 enzyme or other lysyl hydroxylase enzyme, or a fragment or modified form of these having lysyl hydroxylase or glycosyltransferase activity or both activities, or
agents having an effect to the activity of lysyl hydroxylase 3 enzyme or other lysyl hydroxylase enzyme, or a fragment or modified form of these having lysyl hydroxylase or glycosyltransferase activity or both activities; or
a nucleic acid sequence encoding lysyl hydroxylase 3 enzyme or other lysyl hydroxylase enzyme, or a fragment or modified form of these having lysyl hydroxylase or glycosyltransferase activity or both activities; and optionally
functional, preferably oligomerized form of non-collagenous protein; or
a nucleic acid sequence encoding non-collagenous protein.

A pharmaceutical composition according to the invention may comprise any of the above mentioned products, alone or as various combinations.

The present invention provides a method for treating a disorder or condition which is associated with abnormal amount of non-collagenous protein, or abnormal oligomerization or dysfunction of non-collagenous protein in the blood circulation and/or tissue of a patient. The method may comprise administering any of the above mentioned products alone or as various combinations.

The above mentioned products can thus be used for adjusting functional form of the non-collagenous protein in the blood circulation and/or tissue of the patient substantially to the level it is in the blood circulation and/or tissue of a healthy person.

Within the scope of the present invention is a treatment which comprises that the amount or activities of lysyl hydroxylase and/or glycosyltransferase of LH3 (or other LH having these activities) are adjusted in the blood circulation and/or tissue of the patient to a level of a healthy person or to a level, which is more appropriate to the patient and which is therapeutically effective.

Based on the specific activity of purified human recombinant LH3 it is estimated that the amount of LH3 present in human and mouse sera corresponds to about 20 and 70 ng/ml, respectively (Salo et al. 2006). The intracellular amount of LH3 may e.g. 10-fold compared to sera, depending on the partitioning in various tissue types. Wang et al. 2002(b) explains the in vitro activity of LH3 enzyme. In the galactosylation and glucosylation reactions in vitro, 1 ng recombinant LH3 is able to transfer about 1.6 pmole galactose and about 20 pmole glucose in 1 h at 37° C. Similarly, 1 ng of LH3 with LH activity is able to decarboxylate about 0.8 pmol of 2-oxoglutarate.

A therapeutically effective amount or activity means an amount or activity, which is capable of improving the condition of the patient. Preferably it means an amount or activity of lysyl hydroxylase 3 (LH3) or other lysyl hydroxylase (LH) having lysyl hydroxylase and glycosyltransferase (GT and GGT) activities or a fragment or modified form of these having all or one or two of these activities (here for conciseness “LH3 or other LH having lysyl hydroxylase and/or glycosyltransferase activities”, capable of modifying non-collagenous protein to HMW or other functional form.

A method for diagnosing any disorder or condition, which is associated with abnormal amount of a non-collagenous protein, or abnormal oligomerization or dysfunction of a non-collagenous protein in the blood circulation and/or tissue of a patient, comprises that the amount or activities of lysyl hydroxylase or glycosyltransferase of LH3 or both activities in the blood circulation and/or tissue of the patient are compared with their level in the blood circulation and/or tissue of a healthy person or a person having the amount or activity of these enzymes on more appropriate level.

The reason for a disorder or condition related to non-collagenous proteins may be abnormal amount of a specific non-collagenous protein, abnormal oligomerization or dysfunction of a non-collagenous protein. The disorder or condition to be treated may thus be reflected for example in a decrease in the amount of total non-collagenous protein and/or its HMW oligomers or other functional form, or in a decrease of the ratio of HMW oligomers or other functional form concentration to total non-collagenous protein concentration in the blood circulation, and/or tissue of the patient.

According to one embodiment of the invention the treatment comprises that an effective amount of LH3 or LH having lysyl hydroxylase or glycosyltransferase activity or both activities, or a fragment or modified form of LH3 or LH having lysyl hydroxylase or glycosyltransferase activity or both activities, is/are administered to the blood circulation of a patient.

According to another embodiment of the invention the treatment comprises that an effective amount of LH3 or LH having lysyl hydroxylase or glycosyltransferase activity or both activities, or a fragment or modified form of LH3 or LH having lysyl hydroxylase or glycosyltransferase activity or both activities is/are administered to the cells or tissue of a patient. If the non-collagenous protein is adiponectin the tissue is preferably adipose tissue.

According to a third embodiment of the invention the treatment comprises that an effective amount of the non-collagenous protein, preferably in HMW or other functional form of the non-collagenous protein responsible for the disorder or condition to be treated is administered to the patient.

According to a fourth embodiment of the invention the treatment comprises that a nucleic acid sequence encoding LH3 or LH or a fragment of LH3 or LH having lysyl hydroxylase or glycosyltransferase activity or both activities is introduced to and expressed in the cells or tissues of the patient in the body of the patient to produce LH3 or LH enzyme or a fragment of LH3 or LH in the cells or tissues.

A nucleic acid sequence encoding non-collagenous protein responsible for the disorder or condition to be treated may be introduced and expressed in the cells or tissues of the patient in the body of the patient to produce said non-collagenous protein in said cells or tissues. If the non-collagenous protein is adiponectin, the cells or tissues are adipose cells or adipose tissue. The expression is preferably carried out in the presence of LH3 or LH or a fragment or modified form of LH3 or LH having lysyl hydroxylase or glycosyltransferase activity or both activities. LH3 or LH may be administrated as protein or a nucleic acid sequence encoding LH3 or LH having lysyl hydroxylase or glycosyltransferase activity or both activities may be introduced and expressed in the cells or tissues of the patient.

A nucleic acid sequence encoding LH3 or LH or a fragment or modified form of LH3 or LH having lysyl hydroxylase or glycosyltransferase activity or both activities may be introduced and expressed in the cells or tissues of a patient. Non-collagenous protein responsible for the disorder or condition to be treated may be administered as protein or a nucleic acid sequence encoding non-collagenous protein may be introduced and expressed in the cells or tissues of the patient. If the non-collagenous protein is adiponectin, the cells or tissues are adipose cells or adipose tissue.

Non-collageous protein may be recombinantly produced in a cell or tissue culture in the presence of LH3 or LH having lysyl hydroxylase and/or glycosyltransferase activities, or it may be synthetically produced.

The present invention provides a pharmaceutical composition comprising LH3 or LH having lysyl hydroxylase or glycosyltransferase activity or both activities, or a fragment or modified form of LH3 or LH having lysyl hydroxylase or glycosyltransferase activity or both activities and optionally a non-collagenous protein and optionally a pharmaceutically acceptable carrier.

The present invention provides also a pharmaceutical composition comprising a nucleic acid sequence encoding LH3 or LH having lysyl hydroxylase or glycosyltransferase activity or both activities, or a fragment or modified form of LH3 or LH having lysyl hydroxylase or glycosyltransferase activity or both activities and optionally a nucleic acid sequence encoding a non-collagenous protein and optionally a pharmaceutically acceptable carrier.

The present invention comprises also the use of any of the above listed products for preparing a pharmaceutical product, which can be used in diagnosing or treating any disorders or conditions associated with abnormal amount of non-collagenous protein, or abnormal oligomerization or dysfunction of non-collagenous protein in the blood circulation and/or tissue of a patient.

LH3 or LH enzyme or non-collagenous protein and/or HMW or other functional form of the non-collagenous protein can be administrated to the blood circulation and/or tissue of a patient by any suitable method known in the art. A suitable amount of LH3 or LH enzyme or non-collagenous protein and/or HMW or other functional form of the non-collagenous protein is an amount which raises or maintains the amount in blood and/or tissue of the patient to or in the level of the enzyme or protein in a healthy person and/or which has advantageous effects to the patient. The protein/enzyme can be targeted to tissue by any suitable method. Such methods are at present well known to a person skilled in the art.

A nucleic acid sequence encoding LH3 or LH enzyme or non-collagenous protein can be administered to tissue of a patient by any gene delivery methods known in the art.

The product to be administrated may comprise polypeptides, antibodies, or polynucleotides including ribozymes or antisense nucleotides. These may be administered directly to the subject (e.g., as polynucleotide or polypeptides); by parenteral injection, e.g., subcutaneously, intraperitoneally, intravenously or intramuscularly, or to the interstitial space of a tissue; by oral and pulmonary administration, or by suppositories, transdermal applications, needles, gene guns, or hyposprays. These may be delivered ex vivo, to cells derived from the subject (e.g., as in ex vivo gene therapy), by delivery of nucleic acids (into cells) for both ex vivo and in vitro applications for example: dextran-mediated transfection, calcium phosphate precipitation, polybrene mediated transfection, protoplast fusion, electroporation, encapsulation of the polynucleotide(s) in liposomes, direct microinjection of the DNA into nuclei. Ex vivo delivery and reimplantation of transformed cells into a subject are known in the art and described in e.g., International Publication No. WO 93/14778.

Administration of polynucleotide includes local or systemic administration by injection, oral administration, particle gun, catheterized administration or topical administration. Polynucleotide composition may contain an expression construct comprising a promoter operably linked to a polynucleotide. Targeted delivery of compositions containing an antisense polynucleotide, sub genomic polynucleotides, or antibodies to specific tissues may be receptor-mediated.

Polynucleotides and polypeptides of the present invention can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Viral-based vectors can be used for delivery of a desired polynucleotide and expression in a desired cell. Examples of viral-based vehicles include for recombinant retroviruses, alphavirus-based vectors (e.g., Sindbis virus vectors, Semliki forest virus), Ross River virus, Venezuelan equine encephalitis virus, adeno-associated virus (AAV) vectors, and administration of DNA linked to killed adenovirus. Non-viral delivery vehicles comprise polycationic condensed DNA linked or unlinked to killed adenovirus alone, eukaryotic cell delivery vehicles cells, nucleic charge neutralization or fusion with cell membranes, naked DNA can also be employed and liposomes.

Examples of mechanical delivery systems are for example the approach described in Woffendin et al., Proc. Natl. Acad. Sci. USA (1994) 91(24):11581, deposition of photopolymerized hydrogel materials and use of ionizing radiation.

Conventional methods for gene delivery are for example hand-held gene transfer particle gun and ionizing radiation for activating transferred gene.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration.

Various methods for injection or infusion are parenteral administration, such as, for example, by intra-articular (in the joints), subcutaneous, intramuscular, intravenous, intradermal, intrathoracially, intrathecal and epidural, intravesicular, intraperitoneally, intranasally, intracerebroventricularly or subdermal. Various transdermal routes of administration are for dermal or skin patches, inhalation, aerosols, implants, oral, rectal, buccal and sublingual.

Various methods for topical administration are for example, as a cream, ointment, gel, spray, or aqueous solutions, oily solutions, emulsions or suspensions. Various nasal administration methods are for example, as a nasal spray, nasal drops, or dry powder.

Various suppositorial (vaginal or rectal administration) are for example insufflation, local administration.

In the pharmaceutical compositions of the present invention for oral, sublingual, subcutaneous, intramuscular, intravenous, transdermal, local or rectal administration, the active principle, by itself or in association with another active principle, can be administered to humans in unit forms of administration mixed with conventional pharmaceutical carriers. The appropriate unit forms of administration include oral forms such as tablets, gelatin capsules, powders, granules and solutions or suspensions to be taken orally, sublingual and buccal forms of administration, aerosols, implants, subcutaneous, intramuscular, intravenous, intranasal or intraocular forms of administration and rectal forms of administration.

The compounds described herein may be provided or delivered in a form suitable for oral use, for example in a tablet, lozenge, hard and soft capsule, aqueous solution, oily solution, emulsion, and suspension. Formulations suitable for oral administration comprise liquid solutions, such as an effective amount of the packaged nucleic acid suspended in diluents, such as water, saline or PEG 400; capsules, sachets or tablets, each containing a predetermined amount of the active ingredient, as liquids, solids, granules or gelatin; suspensions in an appropriate liquid; and suitable emulsions. Thickeners, flavorings, diluents, emulsifiers, dispersing aids or binders may be desirable.

The term “therapeutically effective amount” as used herein refers to an amount of a therapeutic agent to treat, ameliorate, or prevent a desired disease, disorder or condition, or to exhibit a detectable therapeutic or preventative effect. The effect can be detected by, for example, chemical markers or antigen levels. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation is determined by routine experimentation and is within the judgment of the clinician. The dosage will depend on the route of administration, the severity of the disease, age and weight of the patient and other factors normally considered by the attending physician, when determining the individual regimen and dosage level as the most appropriate for a particular patient. Dosage treatment can be a single dose schedule or a multiple dose schedule.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles.

Typically, the therapeutic compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like.

Many formulations for controlled or sustained release are known and commercially available. These are typically formed of a biodegradable polymeric material or polymeric material which is fabricated to provide slow release of the drug. The controlled release composition is preferably a microparticle formulation. The microparticles preferably include a biodegradable, biocompatible polymer such as polylactide that degrades by hydrolysis. In addition to microparticle systems, other controlled-release injectable or implantable formulations can be used. Both degradable and non-degradable excipients can be used in the formulation of injectable or implantable controlled-release formulations, although degradable excipients are preferred. As used herein, the term “microparticles” includes microspheres and microcapsules. The microparticles preferably are biodegradable and biocompatible, and optionally are capable of biodegrading at a controlled rate for delivery of a compound. The particles can be made of a variety of polymeric and non-polymeric materials.

The oligomerization of non-collagenous proteins in cells or tissues outside the body of a patient can be achieved by incubating the cells or tissues with LH or LH3 enzyme or fragment or modified form of these enzymes. The enzyme is used preferably 0.5 to 100 μg/ml, more preferably 1 to 75 μg/ml, typically the amount is 10-50 μg/ml, preferably 20-40 μg/ml, most preferably 30 μg/ml is added to the cell or tissue culture.

By the term “transform” is here meant any method by which a nucleic acid sequence is introduced into a cell or tissue, such as transformation, transfection, electroporation etc.

The enzymes or fragments of enzymes of the invention can be used in various types of growth systems. For example, the enzymes or fragments of enzymes can be attached to a solid or semisolid or liquid material, which can be used to culture cells, such as a microtiter plates (solid material) or a gel system for culturing cells, such as Matrigel™ Basement Membrane Matrix.

The enzymes or fragments of the enzymes of the invention can be used in various types of cell or tissue culture systems.

The invention includes also vectors, preferably expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

The recombinant expression vectors of the invention can be designed for expression of the proteins of the invention in prokaryotic or eukaryotic cells. For example, polypeptides of the invention can be expressed in E. coli, insect cells (e.g., using baculovirus expression vectors), yeast cells or mammalian cells. Suitable host cells are discussed for example in Goeddel, (1990) Gene Expression Technology: Methods in Enzymology 185, Academic Press, San Diego, Calif.

When used in mammalian cells, the expression vector's control functions are often provided by viral regulatory elements. For example, commonly used promoters are derived from polyoma, Adenovirus 2, cytomegalovirus and Simian Virus 40.

The nucleic acid and polypeptides, fragments thereof, as well as antibodies of the invention can be incorporated into pharmaceutical compositions. Such compositions typically include the nucleic acid molecule, protein, antibody, Fab fragment, and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, relieve, alter, alleviate, ameliorate, remedy, improve or affect the disease, the symptoms of disease or the predisposition toward disease. A therapeutic agent includes, but is not limited to, small molecules, antibodies, peptides, ribozymes and antisense oligonucleotides.

### EXAMPLES

### Example 1

### Adiponectin Measurements by Enzyme-Linked Immunosorbent Assay (ELISA) and Quantification of Oligomeric Forms of Adiponectin by Gel Filtration Chromatography

Total adiponectin level was measured by using specific enzyme-linked immunosorbent assay (ELISA) for mouse adiponectin (Xu et al. 2005) from the LH mutant mice, where the lysyl hydroxylase (LH) activity of LH3 has been specifically mutated (Ruotsalainen et al. 2006). ELISA measurements were done from the serum and adipose tissue homogenate of 3.5, 8 and 10 months old male and 9 and 10 months old female LH mutant mice. Adipose tissue was homogenized into 25 mM Hepes pH 7.5, 5 mM EDTA, 5 mM EGTA, 100 mM NaCl, 1% glycerol, 1% Triton X-100 buffer including Complete protease inhibitor cocktail (Roche) and disrupted by brief sonication. Cell debris was removed by centrifugation before analysis.

In order to analyze the effect of changed LH3 activities on the distribution of different oligomeric forms in LH mutant serum and adipose tissue homogenate and in LH3 knockout MEF cell culture medium transfected with human adiponectin expression construct, total adiponectin in serum/homogenate/medium was fractionated by gel filtration chromatography using HiLoad 16/60 Superdex 200 or Superdex 200 10/300 GL column (Wang et al., 2006) and the concentration of each oligomeric form of adiponectin was determined by mouse adiponectin ELISA or by immunoblot analysis (Xu et al. 2005).

### Quantitative RT-PCR

For gene expression analysis, RNA was isolated from epididymal adipose tissue of LH mutant and wild type mice using Trizol reagent (Invitrogen). The cDNA first strand was synthesized from 0.5 μg of isolated total RNA using a Cloned AMV first-strand cDNA synthesis kit (Invitrogen). Real-time quantitative RT-PCR was performed using a TaqMan® Universal PCR Master Mix (Applied Biosystems) and an ABI 7700 Sequence Detection System (Applied Biosystems). Adiponectin was amplified using adiponectin specific TaqMan gene expression assay Mm00456425_ml (Applied Biosystems). The results were normalized to 18S rRNA quantified from the same samples using forward and reverse primers and probe 5′-TGGTTGCAAAGCTGAAACTTAAAG-3′ (SEQ ID NO:54), 5′-AGTCAAATTAAGCCGCAGGC-3′(SEQ ID NO:55) and 5′-CCTGGTGGTGCCCTTCCGTCA-3′ (SEQ ID NO:56), respectively.

### Immunoblot Analysis

Adipose tissue was homogenized into 50 mM Tris-HCl pH 7.4, 150 mM NaCl, 1% Triton X-100, 1% Igepal, 0.1% SDS buffer including Complete protease inhibitor cocktail (Roche) and disrupted by brief sonication. The cell debris was removed by centrifugation. The protein concentrations were measured with a Bradford assay (BioRad) and 50 μg or 41 μg or 8 μg of soluble protein were loaded into the gel. For mouse serum analysis equal volumes (10 μl of 1/300 diluted serum) of LH mutant and wild type serum were loaded into the gel, and for determination of molecular weight of adiponectin 5 μl of wild type and 10 μl of LH mutant 1/300 diluted serum were loaded into gel. The reduced denaturated proteins were separated by 12 or 15% SDS-PAGE and blotted to nitrocellulose or PVDF membrane. The membranes were blocked with 5% milk powder in TBST and incubated with rabbit anti-adiponectin (Affinity BioReagents) followed by a horse radish peroxidase-conjugated anti-rabbit IgG (P.A.R.I.S.). For anti-FLAG M2 antibody (Sigma) membranes were blocked with 3% milk powder in TBS, incubated with FLAG antibody followed by a horse radish peroxidase-conjugated anti-mouse IgG (P.A.R.I.S.). Immunocomplexes were visualized using ECL+ detection system (Amersham Biosciences) and LAS-3000 imaging system (Fujifilm Life Science) or exposed to Biomax MS film (Kodak). Quantification of adiponectin levels was done using Quantity One® software (BioRad) or ImageQuant TL (GE Healthcare).

### Production of Recombinant Adiponectin

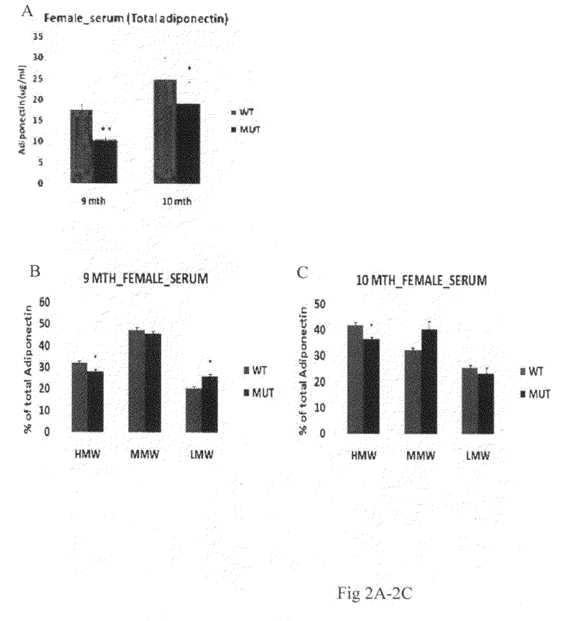

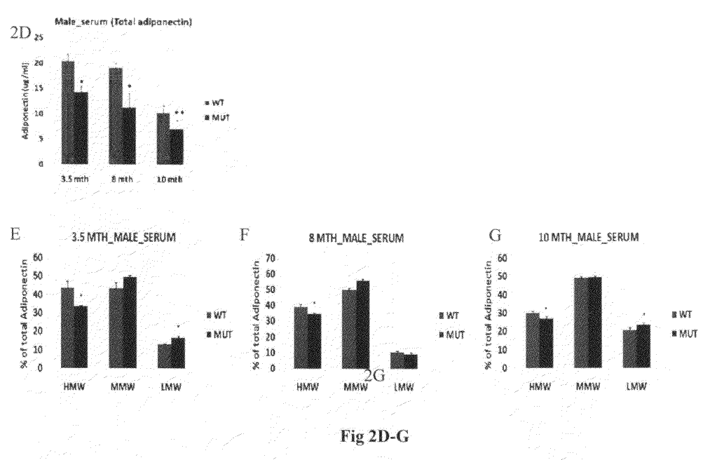

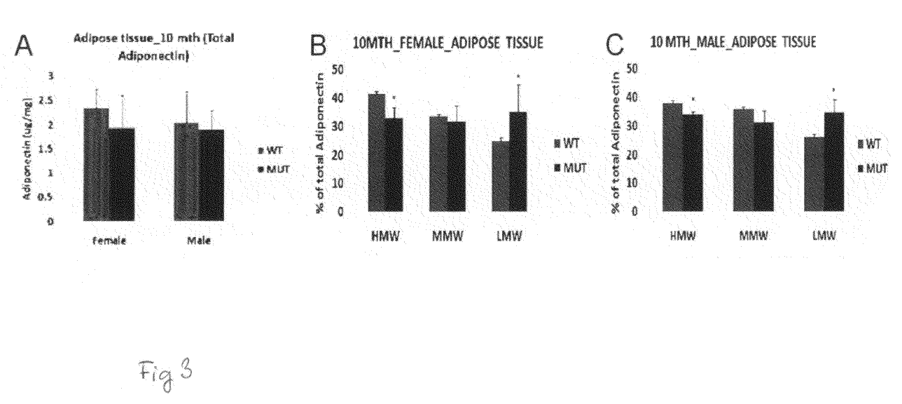

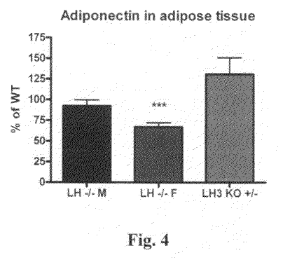

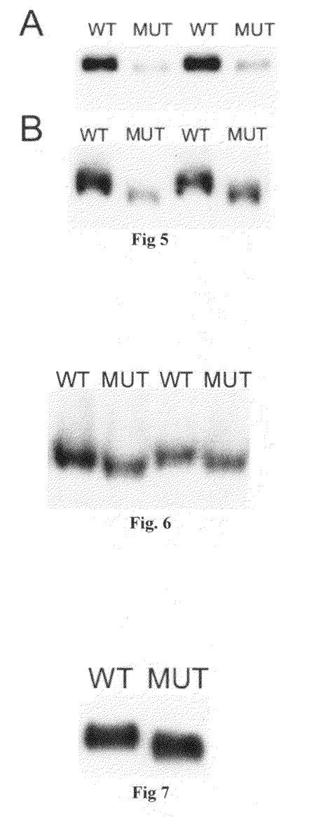

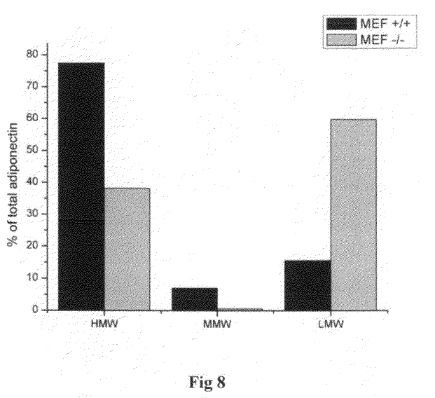

In mammalian expression construct encoding human adiponectin ordered from the gene synthesis service of Eurofins MWG Operon full-length adiponectin is cloned into pcDNA3.1 (+) vector with a FLAG epitope tag at its C-terminus. The expression vector was transfected into LH mutant and wild type skin fibroblasts derived from the skin of newborn pups, or LH3 knockout and wild type mouse embryonic fibroblasts (MEFs) derived from E8.5 embryos. Transfections were done using Nucleofector® technology with MEF Nucleofector kit 1 (Lonza). For immunoblot analysis produced adiponectin was first immunoprecipitated from cell culture medium using Anti-FLAG M2 affinity gel (Sigma) and analyzed with immunoblot using anti-FLAG M2 antibody (as described above). For fractionation of oligomeric forms of adiponectin cell culture medium was concentrated to at least 13^{th} part of the starting volume. Results from LH mutant mice show that LH3 regulates the production of adiponectin in several levels:
1) Adiponectin lacks lysine modifications
To investigate whether LH3 protein affects directly the gene expression, the mRNA level of adiponectin was measured from adipose tissues of LH mutant mice using quantitative real time PCR. The expression level of adiponectin was normal in epididymal adipose tissue of LH mutant male and female mice. This result indicates that LH3 does not affect the expression of adiponectin. Total adiponectin level and oligomeric complexes were analysed from epididymal adipose tissue. In adipose tissue, total adiponectin level was not significantly reduced in 10 months old female and male LH mutant mice (FIG. 3A) in ELISA analysis. In immunoblot analysis the monomeric adiponectin protein level was quantified to be in females 67% and in males 92% of control in 10 month old mice (FIG. 4B), and 92% of control in male 6 months old mice (FIG. 6). Immunoblot analysis indicated a mobility shift between wild type and LH mutant adiponectin in 6 months old male mice (FIG. 6); the size difference corresponds with loss of 1-2 Glc-Gal-Hyl residues. Recombinant adiponectin produced in skin fibroblasts derived from LH mutant newborn pup migrated also faster on SDS-PAGE when compared with adiponectin produced in wild type cells (FIG. 7); the size difference corresponds with the loss of 2 Glc-Gal-Hyl residues. Together these results suggest that adiponectin is produced in almost normal level in adipose tissue, but all of its lysine residues are not properly hydroxylated and glycosylated.
2) Disturbed oligomerization of adiponectin in adipose tissue
The fractionation of oligomeric forms of adiponectin showed altered distribution of oligomeric forms in adipose tissue of LH mutants. HMW was significantly decreased and LMW was significantly increased in both sexes (FIGS. 3B and C) suggesting that the level of glycosylated hydroxylysines in adiponectin is altered in LH mutant mice. These results indicate that LH3 is really the enzyme catalysing the posttranslational modifications in adiponectin, and thus regulate the formation of different oligomeric forms of adiponectin.
3) Delayed secretion of adiponectin
In serum, total adiponectin levels were significantly decreased in females (FIG. 2A) and males (FIG. 2D) in all age groups measured, when compared with the wild type. The amount difference was also clearly seen in adiponectin immunoblot analysis (FIG. 5A). Immunoblot analysis indicated a clear mobility shift between wild type and LH mutant adiponectin (FIG. 5B); size difference corresponds with a loss of 3 Glc-Gal-Hyl residues. Fractionation of serum adiponectin showed that the HMW form was reduced both in the females (FIGS. 2B and C) and males (FIGS. 2E, F and G) in all age groups and accordingly the levels of MMW and/or LMW were increased. The evident reduction of total adiponectin level in serum and nearly normal level of adiponectin in adipose tissue indicate that the secretion of adiponectin and, accordingly, formation or secretion of adiponectin oligomers is affected due to lack of lysine modifications.
4) Disturbed adiponectin oligomerization in LH3 knockout cells
The distribution of recombinant adiponectin produced in LH3 knockout MEFs was altered when compared with adiponectin produced in wild type MEFs (FIG. 8). The amount of HMW and MMW adiponectin was reduced, whereas the amount of LMW adiponectin was increased. The data confirms the finding that LH3 catalyzes the lysine modifications of adiponectin and thus affects the oligomerization of adiponectin.

### Example 2

Recombinant adiponectin is produced in the presence of LH3 to synthesize HMW form of adiponectin in cellulo. Recombinant adiponectin is produced as FLAG fusion protein in a suitable mammalian expression system having LH3 or LH added into the medium as a recombinant protein, co-transfected or stably transfected in mammalian cells. Recombinant LH3 is produced as a full length e.g. His-tag fusion protein having all three enzyme activities in insect cells using Baculo virus expression system or in eukaryotic cells with mammalian expression systems, and purified with nickel affinity column. LH3 is produced as LH3 fragment having the activity required for adiponectin oligomerization.

HMW form or adiponectin is administrated to the patient orally, intravenously, intramuscularly, subcutaneously or by direct adipose tissue injection.

### Example 3

Purified recombinant LH3 or fragment or modified form thereof is administered directly into patient by intravenous, intramuscular, subcutaneous or direct adipose tissue injection or using gene therapeutic methods into the adipose tissue if the LH3 level is decreased in patient. Alternatively agents increasing the activity or amount of LH3 are used to enhance oligomerization of adiponectin in insulin resistant states. These agents are administered orally, intravenously, intramuscularly, subcutaneously or by direct adipose tissue injection.

### LITERATURE

Heise C T, et al. (2000) Impaired secretion of rat mannose-binding protein resulting from mutations in the collagen-like domain. J. Immunol. 165(3):1403-9.
Kivirikko and Myllylä (1979) Collagen glycosyltransferases. Int Rev Connect Tissue Res 8:23-72.
Kivirikko and Myllylä (1980) Hydroxylation of prolyl and lysyl residues. In Freeman R B & Hawkins H C (eds) The Enzymology of post-translational modification of proteins. Academic Press, London, 1980, 53-104.
Kobayashi H, et al. (2004) Selective suppression of endothelial cell apoptosis by the high molecular weight form of adiponectin. Circ Res 94(4): e27-31.
Myllylä R, et al. (2007) Expanding the lysyl hydroxylase toolbox: New insights into the localization and activities of lysyl hydroxylase 3 (LH3). J Cell Physiol. 212(2):323-9.
Myllylä, R. et al. (1975) Assay of collagen-galactosyltransferase and collagen-glucosyltransferase activities and preliminary characterization of enzymic reactions with transferases from chick-embryo cartilage. Eur J Biochem. 52(3):401-10.
Nedvídková J et al. (2005) Adiponectin, an adipocyte-derived protein. Physiol Res 54(2):133-40.
Pajvani U B, et al. (2004) Complex distribution, not absolute amount of adiponectin, correlates with thiazolidinedione-mediated improvement in insulin sensitivity. J Biol Chem 279(13): 12152-62.
Richards A A, et al. (2006) Adiponectin multimerization is dependent on conserved lysines in the collagenous domain: evidence for regulation of multimerization by alterations in posttranslational modifications. Mol Endocrinol 20(7): 1673-87.
Ruotsalainen H, et al. (2006) Glycosylation catalyzed by lysyl hydroxylase 3 is essential for basement membranes. J Cell Sci 119 (Pt 4): 625-35.
Salo et al. (2006) Lysyl hydroxylase 3 (LH3) modifies proteins in the extracellular space, a novel mechanism for matrix remodelling. J Cell Phys 207: 644-653
Salo et al (2008) A connective tissue disorder caused by mutations of the lysyl hydroxylase 3 gene. Am J Hum Genet., 83, 495-503.
Sipilä L, et al. (2007) Secretion and assembly of type IV and VI collagens depend on glycosylation of hydroxylysines. J Biol Chem. 282(46):33381-8.
Szmitko P E, et al. (2007) Adiponectin and Cardiovascular Disease. Am J Physiol Heart Circ Physiol. 292(4):H1655-63.
Tilg H & Moschen A R (2006) Adipocytokines: mediators linking adipose tissue, inflammation and immunity. Nat Rev Immunol 6(10): 772-83.
Ünligil U M, et al. (2000) X-ray crystal structure of rabbit N-acetylglucosaminyltransferase I: catalytic mechanism and a new protein superfamily. EMBO J. 19(20): 5269-5280.
Wang C, et al. (2002b) The third activity for lysyl hydroxylase 3: galactosylation of hydroxylysyl residues in collagens in vitro. Matrix Biol 21: 559-566.
Wang C, et al. (2002c) Identification of amino acids important for the catalytic activity of the collagen glucosyltransferase associated with the multifunctional lysyl hydroxylase 3 (LH3). J Biol Chem 277: 18568-18573.
Wang Y, et al. (2002a) Hydroxylation and glycosylation of the four conserved lysine residues in the collagenous domain of adiponectin. Potential role in the modulation of its insulin-sensitizing activity. J Biol Chem 277(22): 19521-9.
Wang Y, et al. (2006) Post-translational modifications of the four conserved lysine residues within the collagenous domain of adiponectin are required for the formation of its high molecular weight oligomeric complex. J Biol Chem 281(24): 16391-16400.
Wang Y, et al. (2008) Post-translational modifications of adiponectin: mechanisms and functional implications. Biochem J. 409(3):623-33.
Xu, A. et al. (2005) Testosterone selectively reduces the high molecular weight form of adiponectin by inhibiting its secretion from adipocytes. J Biol Chem, 280, 18073-18080.

What is claimed is:

1. A method to treat a disorder or a condition, which is associated with abnormal amount of non-collagenous protein, or abnormal oligomerization or dysfunction of non-collagenous protein in blood circulation and/or tissue of a patient, wherein said method comprises the steps of:
a) Determining level of functional form of the non-collagenous protein in the blood circulation and/or tissue of a healthy person and of the patient;
b) Adjusting the level of functional form of the non-collagenous protein in the blood circulation and/or tissue of the patient substantially to the level it is in the blood circulation and/or tissue of a healthy person, by using lysyl hydroxylase and/or glycosyltransferase activity, whereby the non-collagenous protein is modified to high molecular weight multimer HMW or other functional form.

2. The method according to claim 1, wherein in step b) lysyl hydroxylase 3 (LH3) or other lysyl hydroxylase (LH) having lysyl hydroxylase and glycosyltransferase (GT and GGT) activities, or a fragment or modified form of LH3 or LH having at least one of the activities, is used to modify the non-collagenous protein in the body of the patient or in a cell or tissue culture producing the non-collagenous protein.

3. The method according to claim 1, wherein the non-collagenous protein has collagenous domain with at least 6 Xaa-Yaa-Gly repeats, wherein Xaa and Yaa are any amino acids.

4. The method according to claim 3, wherein Yaa is 4-hydroxyproline, hydroxylysine, galactosyl hydroxylysine or glucosylgalactosyl hydroxylysine, and Xaa is proline.

5. The method according to claim 1, wherein the non-collagenous protein is selected from the group of proteins consisting of
a protein comprising glucosylgalactosylhydroxylysine,
a protein comprising hydroxylysine, and
a protein having a lysine in the Yaa position in the Xaa-Yaa-Gly repeat.

6. The method of claim 5, wherein the protein comprises glucosylgalactosylhydroxylysine and it is further selected from the group consisting of adiponectin, mannan binding lectin, C1q subcomponent of complement activation, surfactant protein D and collectin 43.

7. The method of claim 5, wherein the protein comprises a hydroxylysine and it is further selected from the group consisting of surfactant protein A, collagenous tail (collagen Q) of asetylcholinesterase or, buturylcholinesterase, conglutinin and collectin 46.

8. The method of claim 5, wherein the protein has a lysine in the Yaa position in the Xaa-Yaa-Gly repeat and the protein is further selected from the group consisting of collectin liver 1 (CL-L1), collectin placental (CL-P1), collectin kidney 1 (CL-K1), macrophage receptor MARCO, macrophage scavenger receptor type I, macrophage scavenger receptor type II, C1q, tumor necrosis factor related protein (C1qTNF) 1, 2, 3, 5, 6, 7, 8, otolin-1, adipoQ-like 1 (AQL1), adipoQ-like 2 (AQL2), gliacolin 1, gliacolin 2, collagen triple helix repeat containing 1, gliomedin, CRF1 and CRF2.

9. A method for treating a disorder or condition, which is associated with abnormal amount of adiponectin, or abnormal oligomerization or dysfunction of adiponectin in blood circulation and/or tissue of a patient, wherein said method comprises the steps of:
a) Determining level of adiponectin or HMW form of adiponectin in the blood circulation and/or tissue of a healthy person and of the patient; and
b) Adjusting the level of adiponectin and/or HMW form of adiponectin in the blood circulation and/or tissue of the patient substantially to the level it is in the blood circulation and/or tissue of a healthy person by using lysyl hydroxylase and/or glycosyltransferase activity or activities to modify adiponectin to HMW form.

10. The method according to claim 9, wherein in step b) lysyl hydroxylase 3 (LH3) or other lysyl hydroxylase (LH) having lysyl hydroxylase and glycosyltransferase (GT and GGT) activities or a fragment or modified form of LH3 or LH having at least one of the activities is used to modify the adiponectin in the body of a patient or in a cell or tissue culture producing adiponectin.

11. The method according to claim 9, wherein the disorder or condition is selected from the group consisting hyperglycemia, insulin resistance, metabolic syndrome associated with insulin resistance, type 2 diabetes mellitus, dyslipemia, obesity, weight gain, metabolic syndrome, hypertension, artherosclerosis, coronary heart disease, ischemic heart disease, inflammation and inflammatory diseases.

12. The method according to claim 2, wherein in step b) a pharmaceutically effective amount of lysyl hydroxylase 3 (LH3) or other lysyl hydroxylase (LH) having lysyl hydroxylase and glycosyltransferase (GT and GGT) activities or a fragment or modified form of LH3 or LH having at least one of the activities is administered to the blood circulation and/or to the cells and/or tissues of the patient.

13. The method according to claim 1, wherein in step b) adjusting the level of functional form of the non-collagenous protein is achieved by administering a pharmaceutically effective amount of the non-collagenous protein to the blood circulation and/or to the cells and/or tissues of the patient in HMW or other functional form.

14. The method according to claim 1, wherein in step b) the treatment comprises that an isolated nucleic acid sequence encoding LH3 or LH having lysyl hydroxylase and glycosyltransferase (GT and GGT) activities or a fragment or modified form of LH3 or LH having one or more of the activities, is introduced to and expressed in cells and/or tissues of the patient to produce LH3 or LH or a fragment of LH3 or LH in the cells and/or tissues.

15. The method according to claim 1, wherein in step b) adjusting the level of functional form of the non-collagenous protein is achieved by introducing and expressing in the cells and/or tissues to the patient a nucleic acid sequence encoding the non-collagenous protein responsible for the disorder or condition to be treated to produce said non-collagenous protein in said cells and/or tissues.

16. The method according to claim 1, wherein the non-collagenous protein is adiponectin.

17. The method according to claims 16, wherein the cells or tissues are adipose cells or adipose tissue.

18. The method according to claim 1, wherein LH3 or LH having lysyl hydroxylase and glycosyltransferase (GT and GGT) activities or a fragment or modified form of LH3 or LHthese having at least one of the activities consists of an amino acid sequence selected from the group consisting of SEQ ID NO: 26 to SEQ ID NO:52, and sequences having at least 80% identity to the sequences SEQ ID NO: 26 to SEQ ID NO: 52.

19. The method according to claim 18, wherein the enzyme or a fragment of the enzyme lacks signal sequence.

20. Lysyl hydroxylase 3 (LH3) or other lysyl hydroxylase (LH) having lysyl hydroxylase and glycosyltransferase (GT and GGT) activities or a fragment or modified form of LH or LH2 having at least one of the activities for treatment of a disorder or condition, which is associated with abnormal amount of non-collagenous protein, or abnormal oligomerization or dysfunction of non-collagenous protein in the blood circulation and/or tissue of a patient.

21. An isolated nucleic acid sequence encoding lysyl hydroxylase 3 (LH3) or other lysyl hydroxylase (LH) having lysyl hydroxylase and glycosyltransferase (GT and GGT) activities or a fragment or modified form of LH3 or LH having at least one of the activities for treatment of a disorder or condition associated with abnormal amount of non-collagenous protein, or abnormal oligomerization or dysfunction of non-collagenous protein in blood circulation and/or tissue of a patient.

22. A method for producing non-collagenous protein in HMW or in other functional form, said method comprising a step of producing the non-collagenous protein in a cell or tissue culture in the presence of LH3 or LH having lysyl hydroxylase and glycosyltransferase (GT and GGT) activities or a fragment or modified form of LH3 or LH having at least one of the activities.

23. The method of claim 22, wherein the non-collagenous protein is adiponectin and the cell or tissue is adipose cells or tissues.

24. The method according to claim 22, wherein the protein is produced by introducing and expressing an isolated nucleic acid sequence encoding the non-collagenous protein in a cell or tissue culture in the presence of LH3 or LH having lysyl hydroxylase and glycosyltransferase (GT and GGT) activities or a fragment or modified form of LH3 or LH having at least one of the activities.

25. The method according to claim 22, wherein the presence of LH3 or LH having lysyl hydroxylase and glycosyltransferase (GT and GGT) activities or a fragment or modified form of LH3 or LH having at least one of the activities is achieved by expressing an isolated nucleic acid sequence encoding LH3 or LH having lysyl hydroxylase and glycosyltransferase (GT and GGT) activities or a fragment or modified form of LH3 or LH having at least one of the activities is introduced and expressed in the cell or tissue culture.

26. The method according to claim 1, wherein the method comprises a step of administering LH3 or LH having lysyl hydroxylase and glycosyltransferase (GT and GGT) activities or a fragment or modified form of LH3 or LH having at least one of the activities, and optionally non-collagenous protein, to the patient.

27. The method according to claim 1, wherein the method comprises a step of administering non-collagenous protein in HMW or in other functional form, and optionally LH3 or LH having lysyl hydroxylase and glycosyltransferase (GT and GGT) activities or a fragment or modified form of LH3 or LH having at least one of the activities to the patient.

28. The method according to claim 1, wherein the method comprises a step of administering a nucleic acid sequence encoding LH3 or LH having lysyl hydroxylase and glycosyltransferase (GT and GGT) activities or a fragment or modified form of LH3 or LH having at least one of the activities, and optionally non-collagenous protein or a nucleic acid sequence encoding non-collagenous protein to the patient.

29. The method according to claim 1, wherein the method comprises a step of administering a nucleic acid sequence encoding non-collagenous protein, and LH 3 or LH having lysyl hydroxylase and glycosyltransferase (GT and GGT) activities or a fragment or modified form of LH3 or LH having at least one of the activities, or a nucleic acid sequence encoding LH3 or LH having lysyl hydroxylase and glycosyltransferase (GT and GGT) activities or a fragment or modified form of LH3 or LH having at least one of the activities, to the patient.

30. A method for preparing a medicament for treatment of a disorder or condition, which is associated with abnormal amount of non-collagenous protein, or abnormal oligomerization or dysfunction of non-collagenous protein in blood circulation and/or tissue of a patient, said method comprising a step of modifying the non-collagenous protein with LH3 or LH enzyme or producing the protein in a cell or tissue culture in presence of LH3 or LH enzyme thereby modifying the non-collagenous protein to HMW or other functional form.

31. A pharmaceutical composition comprising LH3 or LH having lysyl hydroxylase and glycosyltransferase (GT and GGT) activities or a fragment or modified form of LH3 or LH having at least one of the activities, collagenous tail (collagen Q) of asetylcholinesterase and optionally non-collagenous protein and a pharmaceutically acceptable carrier.

32. The pharmaceutical composition of claim 31, wherein the composition further comprises a non-collagenous protein in HMW or other functional form.

33. A pharmaceutical composition comprising an isolated nucleic acid sequence encoding LH3 or LH having lysyl hydroxylase and glycosyltransferase (GT and GGT) activities or a fragment or modified form of LH3 or LH having at least one of the activities, and a pharmaceutically acceptable carrier.

34. The pharmaceutical composition of claim 33, wherein the composition further comprises an isolated nucleic acid sequence encoding non-collagenous protein.

35. A method for diagnosing a disorder or condition associated with abnormal amount of non-collagenous protein, or abnormal oligomerization or dysfunction of non-collagenous protein in blood circulation and/or tissue of a patient, said method comprising the steps of:
a) Determining the amount or activity or activities of lysyl hydroxylase and/or glycosyltransferase in the blood circulation and/or plasma and/or tissue of the patient and a healthy person; and
b) Comparing the amounts or activities determined in the blood circulation and/or plasma and/or tissue of the patient with those of the healthy person.
